SEQUENCE LISTING (25 sequences)

SEQ ID NO: 1 - DNA, 24 bp, Artificial
  Primer oDW95
agatcacataataagcccggaacc

SEQ ID NO: 2 - DNA, 25 bp, Artificial
  Primer oDW06
ccattccttatcgacgagcctttgg

SEQ ID NO: 3 - DNA, 31 bp, Artificial
  oDW73
tcactcgagctgggtcactcagctttttcgt

SEQ ID NO: 4 - DNA, 29 bp, Artificial
  oDW86
caggctatgcaaaaatatttcttattacc

SEQ ID NO: 5 - DNA, 30 bp, Artificial
  Primer oDW43
tacgctccatggagtgctcactgcaagcgc

SEQ ID NO: 6 - DNA, 30 bp, Artificial
  Primer oDW44
tacgctccatggtgtgctcacagcaagcgc

SEQ ID NO: 7 - DNA, 30 bp, Artificial
  Primer oDW45
tacgctccatggagtgctcacagcaagcgc

SEQ ID NO: 8 - DNA, 28 bp, Artificial
  Primer oDW46
ttcaccgtcatcaccgaaacgcgcgagg

SEQ ID NO: 9 - protein, 372 aa, Artificial
  Consensus sequence for the UNC-74/TMX3 family of proteins
FYAPWCXXCKXXXPXXXXVXXXXXXXXXXXXVGXXDXTXXXXXAXXXXVXXXPTIXXXXXXXXXXXXGXRXXXXXXXXXXRXXXXXXXXXXXXXXXXXXXXXXXXXXXXXXGXXXXXXXXXXXXXAXXXXXXXXFXXXXXXXXXXXXXXXXXXXXXVXKXXXXXXXXXXXXXXXXXXXXXXXXXXXWXXXERXXXXXXXXXXXXXXXXXXXKXXXXXVXXXXXXXXXXXXXXXXXXXXXXXXXXXXXXXXXXXXFQFXXXXXXXXXXXXXXXXXXXPXXXXXNXXXXXXXXXXXXXXXXXXXXXXXFXXXXXXXXXXXXXXXXGGXXXXXXXXRXXXXXXXXXXXXXXXXPXXXXXXFGXPXXXXSXXXYXI

SEQ ID NO: 10 - DNA, 1344 bp, Caenorhabditis elegans
  CDS (1)..(1344)
atgcaaaaatatttcttattaccgcttttaagcctctctgtactgttattcgtgtatgataccgaagcaacaaatcctccaacagcagttctcgacttgagcgataaatttttggatgtgaaagatgaaggaatgtggtttgttgaattctacgctccatggtgtgctcactgcaagcgccttcatccagtttgggaccaagttggacatacattgtctgacagcaatttacctatcagagtaggaaagctcgattgcacccgtttcccagcagttgccaataaattgagcattcaaggatatccaacgattttgttcttccgaaacggccatgttattgactacagaggcggaagagagaaggaggctctcgtcagttttgccaaaagatgcgctgcaccaatcatcgaagttataaacgaaaatcaaattgaaaaagtgaagctctccgcacgttctcaaccatcatatgtcttcttcggtacatcttctggaccacttttcgacgcattcaatgaagcagcaagctcaaaattttcagttgctcgtttctattccgttgctccaccagaaaatgatgcctcgttccgccaacgcgtcgctgtattcaaagataatttcgaaatcgaattcaacggagacatcgagaaattgacagaatgggtaaccagagaaagatggccaggattccttcaagcaacttcttcaaatctcgctgaaattggtgcaagtggaaagcttgtggtgttggtggtttctagtgaaagtcacaagtttaacaacacaagccccattcgagaattccacaaaactgccgaagaagcatcgaaagagctccgaaagcatccagatctatggaatcgattccaattcgcatggctcgatgggtcggatctggcttctcaaattcaaatggcagctgtctcggagccacatttgttcattttcaattacaccagttatgaatattatctgagcgaggatgagccaagtcaaatgacaatcaaatctattctgacatttttggaacagacttcagaaggaattgataaggaaactattgttgctttcggaggtcgtcacttgttgacaagaatcaagagaatggctttcgaattgtactggaacattgctcaaatgttcgcaactcagccacttttatcgtcatgcctgttcggagtaccaatcgcatttctcagcatcatctgctatagtatttgctccgctgatttcacagtggatcgtgatgagttctatggcgatgaagacgaactgattgatgacgaggaaggtgaagaaacggaacatccggaaaccgatgatgatcacgaaaaagctgagtga

SEQ ID NO: 11 - protein, 447 aa, Caenorhabditis elegans
MQKYFLLPLLSLSVLLFVYDTEATNPPTAVLDLSDKFLDVKDEGMWFVEFYAPWCAHCKRLHPVWDQVGHTLSDSNLPIRVGKLDCTRFPAVANKLSIQGYPTILFFRNGHVIDYRGGREKEALVSFAKRCAAPIIEVINENQIEKVKLSARSQPSYVFFGTSSGPLFDAFNEAASSKFSVARFYSVAPPENDASFRQRVAVFKDNFEIEFNGDIEKLTEWVTRERWPGFLQATSSNLAEIGASGKLVVLVVSSESHKFNNTSPIREFHKTAEEASKELRKHPDLWNRFQFAWLDGSDLASQIQMAAVSEPHLFIFNYTSYEYYLSEDEPSQMTIKSILTFLEQTSEGIDKETIVAFGGRHLLTRIKRMAFELYWNIAQMFATQPLLSSCLFGVPIAFLSIICYSICSADFTVDRDEFYGDEDELIDDEEGEETEHPETDDDHEKAE

SEQ ID NO: 12 - protein, 422 aa, Mus musculus
MALLRAVVSSVLLSRVLLALVLDLDDSFKDSRMEDVWLVDFYAPWCGYCKKLEPVWEEVGAELSRSGSPVRVGKMDATAYSGMASEFGVRGYPTIKLLKGDLAYNYKGPRTKDDIIEFANRVAGPAVRALPSRQMFEHVLKRHSVLFLYVGGESPLKEKYIEVASELIVYTYFFSASEEVLTEAVVLPELPSVVVFKDASFFTYDEYEDGSLSSWVNRERFQSYLQIDGFTLYELGETGKLVAIAVTDDKDQSDHSSRLKGLIQRVATEHREQFNRDFQFGHMSGNDYINSLIMGEVSIPSIIILNTSNEQYFLPAEPVEDLQQMLQFFSSVLDGSAPAYGGDGIFQRIKRVAYDARSTIMSVFRSSPLLGCFLFGLPLGVISLMCYGICTAESDDGTEDLELMKAEEEEEEERLTEKKKKK

SEQ ID NO: 13 - protein, 399 aa, Mus musculus
FVEDLDDTFKDKRVNDIWLVDFYAPWCGYCKKLEPIWNEVGLELKTSGSPVRVGKMDATAYSGMASEFGVRGYPTIKLLKGDLAYNYKGPRTKDDIIEFANRVAGPTVRALPSKEMFEHMLKRHSVLFVYVGGESPLKEKYSDVASELIVYTYFFSASEQVFPEAKLPAVVVFKDGGYFTYDEYEDASLSSWVNKERFQGYLLIDGFTLYELGETGKLVAIAVIDEKDRAEESDRLKTLMQRVAKEYREHFSRDFQFGHMDGSDYINSLIMGEMPVPSVIILNTSNEQFFLPNEFIGTVEQLVHFINSVLNGSAQAYGGDGFFQKVRRIGFDARSTVMSVFRSSPLLGCFLFGLPLGVISLMCYGICTAESDYSMDDIDAHKRDGLTDEEEEEEEEEEE

SEQ ID NO: 14 - protein, 454 aa, Onchocerca volvulus
MAAWKSWTALRLCATVVVLDMVVCKGFVEDLDESFKENRNDDIWLVDFYAPWCGHCKKLEPIWNEVGLEMKSIGSPVKVGKMDATSYSSIASEFGVRGYPTIKLLKGDLAYNYRGPRTKDDIIEFAHRVSGALIRPLPSQQMFEHMQKRHRVFFVYVGGESPLKEKYIDAASELIVYTYFFSASEEVVPEYVTLKEMPAVLVFKDETYFVYDEYEDGDLSSWINRERFQNYLAMDGFLLYELGDTGKLVALAVIDEKNTSVEHTRLKSIIQEVARDYRDLFHRDFQFGHMDGNDYINTLLMDELTVPTVVVLNTSNQQYFLLDRQIKNVEDMVQFINNILDGTVEAQGGDSILQRLKRIVFDAKSTIVSIFKSSPLMGCFLFGLPLGVISIMCYGIYTADTDGGYIEERYEVSKSENENQEQIEESKEQQEPSSGGSVVPTVQEPKDVLEKKKD

SEQ ID NO: 15 - protein, 454 aa, Caenorhabditis elegans
MAAWKSWAALRLCATVVLLDMVVCKGFVEDLDESFKENRNDDIWLVDFYAPWCGHCKKLEPIWNEVGLEMKSIGSPVKVGKMDATSYSSIASEFGVRGYPTIKLLKGDLAYNHRGPRTKDDIIEFAHRVSGALIRPLPSQQMFEHMQKRHRVFFVYIGGESPLKEKYIDAASELIVYTYFFSASEEVVPEYVTLKEMPAVLVFKDETYFVYDEYEDGDLSSWINRERFQNYLAMDGFLLYELGDTGKLVALAVIDEKNTSVEHTRLKSIIQEVARDYRDLFHRDFQFGHMDGNDYINTLLMDELTVPTVVVLNTSNQQYFLLDRQIKNVEDMVQFINNILDGTVEAQGGDSILQRLKRIVFDAKSTIVSIFKSSPLMGCFLFGLPLGVISIMCYGIYTADTDGGYIEERYEVSKSENENQEQIEESKEQQEPSSGGSVVPTVQEPKDVLEKKKD

SEQ ID NO: 16 - protein, 477 aa, Homo sapiens
GGGSLHYLSLLALIAGFTPRSMANAVGRRSWAALRLCAAVILLDLAVCKGFVEDLNESFKDNRKDDIWLVDFYAPWCGHCKKLEPIWNEVGLEMKSIGSPVKVGKMDATSYSSIASEFGVRGYPTIKLLKGDLAYNYRGPRTKDDIIEFAHRVSGALIRPLPSQQMFDHVRKRHRVFFVYIGGESPLKEKYIDAASELIVYTYFFSASEDVVPEYVTLKEMPAVLVFKDDTYFVYDEYEDGDLSSWISRERFQNYLTMDGFLLYELGDTGKLVAIAVIDEKNTSLEHTRLKSIIQEVARDFRDHFHRDFQFGHMDGNDYINTLLMDELTVPTIVVLNTSNQQYFLLDRHIKDASDMVQFINSILDGTVPAQGGDSIFQRLKRIVFDAKSTIVSIFKSSPLMGCFLFGLPLGVISIMCYGIYTADTDGGYIEERYEVSKSEMENQEQIEESKEQESSSGGSLAPTVQEPKDVLEKKKD

SEQ ID NO: 17 - protein, 461 aa, Oryza sativa
MAAGTLGAGLRLCATGSFRRAACTERRGGRATPACGHAWLGLRAPRPVTSGGFLPTWGLREMAEDRPRWIWNEVGLEMKSIGSPVKVGKMDATSYSSIASEFGVRGYPTIKLLKGDLAYNYRGPRTKDDIIEFAHRVSGALIRPLPSQQMFEHVQKRHRVFFVYIGGESPLKEKYIDAASELIVYTYFFSASEEVVPEYVTLKEMPAVLVFKDETYFIYDEYEDGDLSSWINRERFQNYLTVDGFLLYELGDTGKLVAIAVIDEKNTSIEHTRLKSIIQEVARDYRDQFHRDFQFGHMDGNDYINTLLMDELKVPTVVVLNTSNQQYFLLDRQIKNAEDMVQFINNILDGTVEAQGGDSILQRLKRIVFDAKSTIVSIFKSSPLMGCFLFGLPLGVVSIMCYGIYTADTDGGYIEERYEVSKSEIESQEPTEESKEQEPRSGDALVPTVQGPKDVLEKKKD

SEQ ID NO: 18 - protein, 479 aa, Homo sapiens
MRSEGRSARRRAVSPSGRARSPVNMAAMGGRQQCLWAAAVVALALASEAAFVEDLDESFKENRKDDIWLVDFYAPWCGHCKKLEPVWNEVGMEMKNMGSPVKVGKMDATSFSSIASEFGVRGYPTIKLLKGDLAYNYRGPRTKDDIIEFANRVAGPLIRPLPSQHMFEHVRKRHRVLFVYVGGESPLKEKYIEVASELIVYTYFFSASKDVLPEYLTLPELPAVVVFKDGTYFVYDEYEDGDLSSWINRERFQGYLTVDGFTLYELGDTGKLVAIAVIDDKNSSVEHTRLKSIIQEVARDYRDHFHRDFQFGHMDGNDYINSLLMDDLTIPTIVVLNTSNQQYFLPDRHIENTEDMVQFINNILDGTAEAQGGDGVLQRIKRIVYDAKSTVVSVFKSSPLLGCFLFGLPLGVISIMCYGICTADTDGGVDEHEAVKKENSDRELTDDGSEEEQEEENGKYTELSDGELKQKDLLEKKKD

SEQ ID NO: 19 - protein, 452 aa, Homo sapiens
MAAAGLCFILAIVSSTSLLASVPVSALVEDLDDSFKENRKDDIWLVDFYAPWCGHCKKLEPVWNEVGIEIRTSGSPIRVGKIDATVYSSIASEFGVRGFPTIKALKGDMAYNYRGPRTKEDIVEFANRVAGPLIRPLPSQQMFDHVKKRHPVLFVYVGVESTLKEKFIEVASELIVYTYFFSASEDVLPKYVTLNEVPAVLVFKDSTYFVYDEYEDGDLSSWVNKERFEGYLHIDGFTLYELGDTGKLVAVAVIDEKNNSIEHTRIKSIAQDVAKNNRNNFHRDFQFGHMDGNDYINSLLMDELSIPTFVVLNTSNQQYFLPSKHIENPEEMIQFINSILDGTAEAQGGDGILQRIKRVFYDAKSTVVSVFKSSPLLGCFLFGLPLGVISIMCYGICTADTEDGSEEMTRKDVIDQNASDEGSDEEEEKGREITDVSDEDQQEKDFMEKKID

SEQ ID NO: 20 - protein, 447 aa, Homo sapiens; identical to SEQ ID NO: 11

SEQ ID NO: 21 - protein, 441 aa, Homo sapiens
MFLAIVGIVYILLVPEETEAINPPTAVLDLSDKFLDVKDEGMWFVEFYAPWCAHCKRLHPVWDQVGHSLSDSNLQIRVGKLDCTRFPAVANKLGIQGYPTITFFRNGHAIEYRGGREKEALVSFAKRCAAPIIETIKENQVEKVKLSARSQPSYIFFGHSSGPLFDAFNEAANAKFSVARFYTVAPSKEETNFRQRVVVLKDNVEIEFQEDIEALKDWVVRERWPTFVHATSSNLAELGASGKLVVLIVSSESQKFNTTSPVREFHKVAEDASKEMRKHSALWNRFQFAWLDGSDLASQIQMASVSEPHLFVFNYTSYEYYLSEDEPSQMTIKSIFTFLEQTAEGIDKGTIIAFGGRNLLTRMKRMIFELYWNIAQMFATQPLLSSCLFGVPIAFLSIICYSICSADFTVDRDEFYGDEALEDEEEETDTIETDDDHEKAE

SEQ ID NO: 22 - protein, 465 aa, Rattus norvegicus
FNLLHSRHVRLLNERKKTFSTYLAAAAVHLSNPKINKMYTLCKLLIALCCFTTLAHSSRVLELSDRFLDVRNEGQWFVMFYAPWCAHCKKLEPVWALVAQALYNTNIRVGRVDCTRFTAVAQHFKVNAYPTIIFVKGPYDYVYNGERSKEELIHFVNRMSGPPVQLVTRADSIDILKSNNPIFFTYVGKQSGLLWDVFYSAAESYQAHGYFYATSVEIAKRHFDVDTVPAALVYKERSHYYFPYSDNFERIEPAHLNDTLFRWVNEERFATFPKVTRSNIHHLVQTQKYLVLAVVEENKLSEIAAHEQEFRDMVEIFVHKNKHKYHGRFQFGWVGTPELARSIAMDSLPTPHLLVLNASTNEHHIPEDDPLQLTPEAIEIFLDSIHNQTAPTFGGNSLPVRIYRAWFEAKTSLYEMWIGNPVLTTVLFGLPLGFLSLIMYSICCADILDAEEEDDGADQRHEKNE

SEQ ID NO: 23 - protein, 387 aa, Homo sapiens
MFYAPWCAHCKKLEPVWALVAQALYNTNIRVGRVDCTRFTAVAQHFKVNAYPTIIFVKGPYDYVYNGERSKEELIHFVNRMSGPPVQLVTRADSIDILKSNNPIFFTYVGKQSGLLWDVFYSAAESYQAHGYFYATSVEIAKRHFDVDTVPAALVYKERSHYYFPYSDNFERIEPAHLNDTLFRWVNEERFATFPKVTRSNIHHLVQTQKYLVLAVVEENKLSEIAAHEQEFRDMVEIFVHKNKHKYHGRFQFGWVGTPELARSIAMDSLPTPHLLVLNASTNEHHIPEDDPLQLTPEAIEIFLDSIHNQTAPTFGGNSLPVRIYRAWFEAKTSLYEMWIGNPVLTTVLFGLPLGFLSLIMYSICCADILDAEEEDDGADQRHEKNE

SEQ ID NO: 24 - protein, 430 aa, Mus musculus
MSPNSMWIFGLISALLLTLGSTGLSSKVLELSDRFIDVRHEGQWLVMFYAPWCGYCKKTEPIFALVAQALHATNVRVGRLDCTKYPAAAKEFKVRGYPTIMFIKGNMEFTYNGDRGRDELVDYALRMSGPPVQLVTRTESVDMLKGSHTIFFIFVGQQEGVVWDTYYAAAEGYQEHGFFYATSEDIAAQHFDFEKLPAVIVYKEEQHHFYPHGHLAHEMDPNEVNETVFQWVNVERFTLFPKVTRFNIHQLLKTNKYLVLAVVQEDKLNQIATHELEFRDMVEGVIRKHRARYHDKFQFGWIGEPSIAHSIILDQLPTPHLIAINSSTQHHFIPEDDPMQMTPQALHLFLESIRNESAIAYGGDTYFVRLNRALFEVRRALRDMWLGNPVLTTVIFGLPLGFLSLIMYSIFCGDCLVTEEDPDEDHEKKE

SEQ ID NO: 25 - protein, 454 aa, Homo sapiens; identical to SEQ ID NO: 14

(19) United States
(12) Patent Application Publication
(10) Pub. No.: US 2010/0160217 A1
(43) Pub. Date: Jun. 24, 2010

(54) ACETYLYCHOLINE GATED ION CHANNEL CHAPERONS AND METHODS OF USING THE SAME

(75) Inventors: Daniel C Williams, Hamden, CT (US); Erik M. Jorgensen, Salt Lake City, UT (US)

Correspondence Address: Ballard Spahr LLP, SUITE 1000, 999 PEACHTREE STREET, ATLANTA, GA, 30309-3915, US

(73) Assignee: UNIVERSITY OF UTAH RESEARCH FOUNDATION

(21) Appl. No.: 11/919,095

(22) PCT Filed: May 4, 2006
(86) PCT No.: PCT/US2006/017430

§ 371 Date: Mar. 4, 2010

Related U.S. Application Data
(60) Provisional application No. 60/678,121, filed on May 4, 2005.

(51) Int. Cl. A61K 38/16; C07K 14/00; C12P 21/06; C12N 5/02; C07H 21/04; C12N 15/63; G01N 33/53

(52) U.S. Cl. 514/12; 530/350; 435/69.1; 435/375; 536/23.5; 435/320.1; 435/325; 435/7.1

(57) ABSTRACT

The present invention provides receptor chaperons and means for producing cells having increased and/or decreased expression of nAChR subunit combinations and/or nAChR subtypes, which provide useful models for investigating pharmacological properties of the receptors and regulation of the binding sites of potential nAChR subtypes.

### PRIORITY CLAIM

This application claims the benefit of the filing date of U.S. Provisional Patent Application Ser. No. 60/678,121, filed May 4, 2005, for “ACETYLYCHOLINE GATED ION CHANNEL CHAPERONS AND METHODS OF USING THE SAME”, the contents of which are incorporated by this reference.

### TECHNICAL FIELD

This invention relates to biotechnology, and more particularly to receptor chaperons and methods of using the same.

### BACKGROUND

Nicotinic acetylcholine receptors (nAChRs) are ligand gated ion channels that mediate fast excitatory neurotransmission in the central and peripheral nervous system. nAChRs are distinct from metabotrophic receptors, including the muscarinic acetylcholine receptor, in that they are pentameric integral membrane proteins that form a cation selective channel gated by acetylcholine (Changeux et al., 1984; Karlin and Akabas, 1995). These channels are tightly clustered at the postsynaptic region and modulate the postsynaptic membrane potential in response to presynaptic release of acetylcholine. Adult mammalian muscle type nAChRs are heteropentamers composed of two α1 subunits and one β1, γ and ε subunit. Each of the subunits have the same membrane topology and are composed of a large extracellular region, a transmembrane region composed of four membrane spanning domains and a cytoplamsic region formed by an intracellular loop between the third and fourth membrane spanning domains. (Corringer et al., 2000; Unwin, 2005). The subunits of mature pentameric nAChRs are arranged with a five-fold axis of symmetry with the second transmembrane spanning region lining the central pore of the ion channel (Akabas et al., 1994; Imoto et al., 1988). In addition to an invariant stoichometry, the circular arrangement of subunits is also fixed in the circular order α γ α β ε. The correct ordered assembly of nAChR subunits is functionally important because the acetylcholine binding site is formed at the interface between α/γ and α/ε subunits (Green and Wanamaker, 1998).

After translation, formation of mature nAChRs occurs in different cellular compartments and is a slow inefficient process (Merlie et al., 1983; Baker et al., 2004). Early steps in the formation of nAChRs occur co-translationally on each subunit and are common among transmembrane proteins of the plasma membrane. These early processing steps include signal peptide cleavage, insertion of transmembrane spanning regions into the membrane in the correct orientation, glycosylation, and disulfide bond formation. After or during processing the individual subunits are assembled into pentamers and assembly is regulated to ensure that the correct composition and arrangement of subunits is achieved. Finally, the mature assembled pentamer is trafficked to the plasma membrane. Trafficking of nAChRs is a regulated process; individual nAChR subunits are not trafficked, but remain in the ER until incorporated into pentamers. In contrast to the structural and functional studies of individual receptor subunits, little is known about the molecules and mechanism that govern formation of nAChRs.

nAChRs belong to a family of ligand gated ion channels that all contain a conserved extracellular cystiene loop (Connolly and Wafford, 2004). The Cys-loop is present on all subunits of the family and is formed by a disulfide bond between two cystine residues separated by 13 amino acids. The Cys-loop family includes receptors for common neurotransmitters including, GABA, glycine and serotonin, as well as uncommon ligands such as histidine and zinc (Davies et al., 2003; Le Novere and Changeux, 2001). The functional significance of this conserved structural feature is not well established. High-resolution structural determination of the nAChR, and the acetylcholine binding protein (AChBP), place the Cys-loop near the extracellular linker between the second and third transmembrane spanning region. In addition, mutation of residues within the Cys-loop of a glycine receptor results in a decrease in receptor activation, suggesting that the Cys-loop may have a functional role in gating. (Brejc et al., 2001; Schofield et al., 2003; Unwin, 2005). However, the presence of the Cys-loop in the AChBP, which does not contain a channel, argues against a functional gating role for the Cys-loop. Other groups assert that formation of the Cys-loop in nAChR subunits plays an essential role in nAChR formation, since mutation of the cysteine residues of the Cys-loop blocks assembly of nAChRs and prevents trafficking (Green and Wanamaker, 1997; Sumikawa and Gehle, 1992). Hence, the functional significance of the Cys-loop remains unclear.

C. elegans is an ideal genetic model system to identify genes whose protein product has a role in nAChR formation (Miller et al., 1996). Cholinergic neurotransmission at the worm neuromuscular junction is mediated by two genetically distinct classes of nAChR based on sensitivity to the anthlementic levamisole (Richmond and Jorgensen, 1999a). Levamisole causes activation of the levamisole sensitive nAChR, but does not affect the levamisole insensitive nAChR. Application of levamisole to nematodes causes chronic activation of levamisole sensitive nAChRs and results in hyper-contraction of body muscles, causing paralysis. Forward genetic screens to isolate mutants resistant to levamisole have led to the identification of four genes (unc-29, unc-38, unc-63, and lev-1) that encode subunits of the levamisole sensitive nAChR. In addition, other genes that do not encode nAChR subunits have been shown to confer levamisole resistance when mutated. The protein products of these genes may be involved in formation of the nAChR (Lewis et al., 1980a; Lewis et al., 1980b).

### DISCLOSURE OF INVENTION

The present invention provides receptor chaperons and means for producing cells having increased and/or decreased expression of nAChR subunit combinations and/or nAChR subtypes, which provide useful compositions and tools for investigating pharmacological properties of the receptors and the regulation of the binding sites of nAChR subtypes. In addition, the invention provides compositions and methods for screening nicotinic compounds.

The invention relates to a receptor chaperon protein or a functional fragment thereof. For example, a polypeptide of SEQ ID NO:9, SEQ ID NO:10, SEQ ID NO:12, or FIG. 10. Optionally, the receptor chaperon of the invention may comprise a mutation in a thioredoxin domain.

The invention also relates to nucleic acid sequences, isolated and/or recombinant, encoding a receptor chaperon protein. The nucleic acid sequences may be in a vector, including an expression vector, and may be introduced into a host cell.

The invention also relates to a method of producing a heterologous receptor in a cell, the method comprising: providing a host cell; introducing a nucleic acid sequence encoding at least one subunit of a heterologous receptor that does not efficiently produce a functional receptor on the surface of the host cell; producing the at least one subunit of a receptor in the host; introducing a nucleic acid sequence encoding a receptor chaperon of the invention into the host cell; producing the receptor chaperon in the host cell; and increasing production of a functional receptor comprising the at least one subunit of a heterologous receptor on the surface of the host cell. The receptor subunit of the method may be a nicotinic Acetylcholine receptor subunit derived from a multicellular organism (a subject), such as a vertebrate, insect, Caenorhabditis or mammal, including a human, cow, or horse.

The invention also relates to a method of reducing or eliminating expression of a receptor on a cell surface by inhibiting a function of a receptor chaperon of the invention in a cell. Such inhibitors may be used to treat a subject, for example, the inhibitor may be made into a medicament for the treatment of a disease characterized by receptor hyperactivity. Alternatively, the inhibitor may be used as an insecticide, particularly, when the inhibitor has a high affinity for an insect receptor chaperon, relative to the affinity for a human and/or mammalian receptor chaperon.

The invention further relates to a method of producing a recombinant nematode nicotinic acetylcholine receptor, comprising culturing a host cell under conditions which permit the expression of UNC-74. The unc-74 gene may be coexpressed with one or more nAChr subunits, preferably the nAChR subunits derived from Caenorhabditis.

The invention further relates to a method of screening for anthelmintic compounds by introducing a receptor chaperon of the invention into a host cell; expressing the receptor chaperon in the host cell; contacting the host cell with a compound to be screened for antihehnintic activity; selecting a compound which interacts with said receptor chaperon; and characterizing the selected compound as an anthelmintic compound.

The invention further relates to a method of controlling parasitic nematode growth in a host, comprising: administering an effective amount of an anthelmintic compound identified by a method of the invention to a subject. The invention further relates to a method of controlling parasitic nematode growth in soil or a crop, comprising: administering an effective amount of an anthelmintic compound identified by a method of the invention to the soil or crop. For example, the invention also relates to methods of screening for neonicotinoids and their use for crop protection by screening for compounds that inhibit or prevent the production of functional nAChRs on the surface of insect cells. Preferably, the compound exhibits selective toxicity toward insects based, at least in part, on a higher affinity for an insect receptor chaperon.

### BRIEF DESCRIPTION OF DRAWINGS

FIG. 1(A) shows images of wild-type and unc-74 on solid media with or without 0.2 mM levamisole. Wild type animals hypercontract and become completely paralyzed after ˜2 hours in the presence of millimolar concentrations of levamisole, whereas unc-74 mutants show no response.

FIG. 1(B) illustrates levamisole resistance curves showing the percent of animals paralyzed at increasing concentrations of levamisole. Wild type animals become paralyzed at ˜0.15 mM levamisole. In contrast, unc-74 mutant animals are completely resistant to all concentrations of levamisole tested. Data represents mean +/− SEM of n=3 plates, minimum of 15 animals scored for each plate.

FIG. 1(C) Illustrates the swimming assay, showing the body bend frequency of animals suspended in liquid media. unc-74 animals have a reduced body bend frequency when compared to wild type (P=0.0002, n=3, each allele) and levamisole sensitive nAChR subunit mutants (P<0.005, n=3, pairwise combination of each allele). Subunit mutants also have a reduced body bend frequency compared to wild type (P<0.005, n=3, unpaired t-test).

FIG. 2 illustrates an electrophysiological analysis of levamisole-sensitive acetylcholine gated ion channels. FIG. 2A illustrates representative traces and peak current amplitude induced by micro-second application of levamisole to voltage clamped muscle cells of dissected animals. FIG. 2B illustrates representative traces and peak current amplitude induced by micro-second application of nicotine to voltage clamped muscle cells of dissected animals. Data presented represent mean and SEM from n=3 different animals.

FIG. 3 shows that levamisole-sensitive acetylcholine gated ion channel subunits are retained in the ER. Stacked serial confocal fluorescent images of animals expressing UNC-38:GFP and stained with antibodies against the presynaptic marker SNB-1. In wild type animals, UNC-38:GFP is synaptic as shown by the juxtaposition with anti-SNB-1 signal in merged images. In contrast, UNC-38:GFP is not synaptic in unc-74 animals, but is retained in intracellular compartments in both muscle cells and neurons. Arrowheads indicate neuronal cell bodies and arrows indicate muscle cell nuclei.

FIGS. 4A and B show that UNC-74 function is specific for nAChRs. FIG. 4(A) illustrates a representative trace and quantification of electrophysiological GABA response on wild type and unc-74 animals. FIG. 4(B) illustrates that UNC-49/GABA_{A} receptors are normal in unc-74 mutant animals. GABA_{A}:GFP staining is localized to discrete punctae along nerve cords in wild type and unc-74(ox167) mutant animals.

FIGS. 5A and B illustrate the cloning, protein structure and conservation of unc-74. FIG. 5(A) illustrates the unc-74 rescuing fragment, showing the gene structure and molecular lesions of different alleles. Numbers indicating base pair position are relative to A of ATG. ox167 is an insertion of Mos1 transposon in exon 5. The rescuing fragment was generated by PCR with oligos oDW06 and oDW05, located at indicated sites. FIG. 5(B) Top illustrates the protein structure of unc-74 with identified domains highlighted and allele disruptions indicated. ox78 (A379 [symbol] T) causes a nonsense change at lysine 95; ox145 (ΔC1117-A1159) is a small 43 base pair deletion in exon 5 that causes a frameshift after glycine 213; x19 (C1319 [symbol] T) results in a nonsense change at arginine 280; ox111 (G2096 [symbol] A) disrupts the splice donor of intron 6 which results in a frameshift starting at S405. FIG. 5(B) Bottom illustrates a sequence alignment, showing conservation of the UNC-74/TMX3 thioredoxin active site and transmembrane spanning region in vertebrates and invertebrates. In the thioredoxin domain, active site cysteine residues are in yellow and in the transmembrane spanning region, conserved nonpolar residues are in red, and conserved polar residues are in magenta.

FIG. 6 shows that unc-74 expression in muscles is necessary and sufficient. FIG. 6A illustrates the swimming assay and FIG. 6B illustrates the levamisole resistance assay. The decrease in body bend frequency and levamisole resistance of unc-74 animals can by rescued by expression of the unc-74 cDNA under the muscle cell myo-3 promoter. Rescue is not seen when unc-74 is expressed in neurons with the pan-neuronal promoter rab-3. 3xPrab-3:unc-74 is an extrachromosomal array generated by injecting a three fold higher concentration of plasmid DNA than was used to generate Prab-3:unc-74. The observed difference between wild type and unc-74; Pmyo-3:unc-74 is significant in the swimming assay (P<0.05, n=3), but not in the levamisole resistance assay (P>0.1, n=3).

FIG. 7 shows that UNC-74 is localized to the ER. FIG. 7(A) is a stacked confocal image of head muscles expressing rescuing UNC-74:GFP and TRAM:CFP, both under control of the muscle cell promoter myo-3. Both fusion proteins are co-localized as shown in the merged images. Similar staining was seen in body wall muscle cells. FIG. 7(B) is a single slice of a individual dorsal body wall muscle cell. Fluorescence from both channels is diffuse throughout this cell in a reticulated pattern and concentrated in a ring around the nucleus.

FIGS. 8A-C demonstrate that the UNC-74 thioredoxin active site is dispensable for function. Site directed mutagenesis was performed to change the UNC-74 thioredoxin active site from CAHC to SAHC, CAHS, or SAHS. Then transgenic animals were generated with wild type, mutant, or empty (oxEx[Marker]) unc-74 plasmids and animals tested for rescue of unc-74ox(78) phenotype. Qualitatively, no difference was seen between animals expressing wild type unc-74 and animals expressing active site mutations when observed moving on solid media. FIG. 8(A) illustrates the swimming assay measuring the frequency of body bends of animals suspended in liquid media. N=3 for each strain. FIG. 8(B) illustrates the tracking assay to measure velocity of animals. N=number of animals. FIG. 8(C) illustrates levamisole resistance assay measuring resistance to 0.15 mM levamisole. N=number of plates. Similar results were obtained for multiple independently-generated extrachromosomal arrays. Data represents mean +/− SEM.

FIG. 9 illustrates a model for the function of UNC-74. Cartoon depicting putative mechanism of UNC-74 function. UNC-74 is hypothesized to keep nAChR subunits that do not have an ER retention motif in the ER. This could be accomplished by (A) retaining subunits in the ER, or (A′) shuttling back and forth between the ER and Golgi and retrieving subunit that have left the ER. Subunits that contain the ER retention motif (red stripe) remain in the ER independently of UNC-74. Subunit assembly (B) occurs in the ER and buries the endogenous ER retention motif on subunits. (C) Assembled pentamers are trafficked to the plasma membrane.

FIG. 10 illustrates the sequence conservation found in the receptor chaperon family of the present invention. Preferred amino acids are shown in lower case, and conservative substitutions are illustrated by representative numbers: 2=a hydrophilic side chain, e.g., Gln or Glu; 3=an OH containing amino acid, e.g., Ser or Thr; 4=a positively charged amino acid, e.g., Lys or Arg; 5=An aromatic amino acid, e.g., Tyr, Phe or Trp; and 6=a non-polar amino acid, e.g., Ile, Val, Met.

### MODES FOR CARRYING OUT THE INVENTION

The invention provides a greater understanding of ligand gated ion channel formation, thereby providing compositions and methods useful in the production of such channels. The molecules of the invention, which have not been identified through traditional biochemical analysis, are involved in nAChR formation.

The invention utilizes the cloning and characterization of unc-74, which encodes the worm homologue of TMX3, a transmembrane thioredoxin domain containing protein (Haugstetter et al., 2005), to describe receptor chaperon proteins involved in the formation of multimeric nAChRs.

The data presented herein describes the cloning and characterization of unc-74, which is believed to be an exemplary member of a class of proteins required for the formation of a specific acetylcholine-gated ion channels. Using a transposon-based mutagenesis approach described in International Patent Publication WO 00/73510 (Dec. 7, 2000), the unc-74 locus was cloned and shown to encode the worm homologue of TMX3, a transmembrane thioredoxin containing protein. Electrophysiological and genetic analysis of unc-74 mutant animals demonstrated that UNC-74 is required for the formation of the levamisole-sensitive nAChR. In the absence of unc-74 function, nAChR subunits are retained in the ER of both muscle and neurons. UNC-74 is expressed in muscle cells and localized to the ER, where it is believed to function in the trafficing of nAChRs to the plasma membrane. However, this function does not require the catalytic activity of its thioredoxin domain.

That the thioredoxin active site of UNC-74 is dispensable for function is discordant with the conservation of the UNC-74/TMX3 thioredoxin domain and active site. These data suggest that UNC-74 may have two functions: the promotion of acetylcholine-gated ion channel formation independently of redox, and a redox-dependent function that is not essential and does not produce a visible phenotype when perturbed. For example, UNC-74/TMX3 could provide a redundant contribution to redox homeostasis in the ER.

The protein structure and sequence conservation of UNC-74/TMX3 homologues suggests that this family of thioredoxins has a similar function in all metazoans. Although mammalian nAChRs are insensitive to levmamisole, they do express a variety of nAChRs that are distinct with respect to subunit composition and activity. For example, mammalian capsaicin-sensitive and -insensitive nAChRs are known. Therefore, mammalian UNC-74/TMX3 are believed to distinguish between different nAChR subtypes and is required for the formation of nAChR. Hence, the addition of UNC-74/TMX3 to a cell is believed to allow or facilitate the formation of receptor subtypes that are refractory to heterologous expression.

TABLE 1
Sequence conservation within different regions of UNC-74

| | Identity | | Similarity | |
|---|---|---|---|---|
| Signal peptide | 5/24 | (0.21) | 12/24 | (0.50) |
| Thioredoxin | 40/108 | (0.37) | 89/108 | (0.82) |
| Central | 62/257 | (0.24) | 156.257 | (0.61) |
| Transmembrane | 11/23 | (0.48) | 21/23 | (0.91) |
| Overall | 132/469 | (0.28) | 306/469 | (0.65) |

The amino acid sequence identity and similarity of different regions of UNC-74 between C. elegans and human homologues. Data presented are the number of identical or similar residues within the listed region/total number of residues within a region (frequency of identical or similar residues). The transmembrane spanning region of UNC-74 contains the highest level of sequence conservation.

Three lines of evidence suggest that the UNC-74 transmembrane spanning domain has a function other than just membrane anchoring. First, there is a high level of sequence conservation within this domain compared to other regions of the protein (Table 1). In general, transmembrane domains do not exhibit high sequence identity between species, but rather are composed of similar hydrophobic residues, which anchor the transmembrane spanning region in the membrane. Signal peptides are analogous to transmembrane spanning regions in that they span the membrane and identical sequence conservation is not required for signal peptide function. Comparison of the identity and similarity between UNC-74 and TMX3 (the human homologue) within different regions of the protein indicates that the transmembrane domain is considerably more conserved than the signal peptide. Second, there is a conserved (G/A)XXX(G/A) motif present in the transmembrane domain. This motif is common within helices of both transmembrane and soluble proteins and is involved in helix-helix interactions (Gerber et al., 2004; Senes et al., 2004). The presence of the small side groups at each end of this motif allows the helix to adopt a conformation ideal for hydrogen bonding between two helices. This is suggestive that the transmembrane region of UNC is able to interact with other proteins via helix-helix interactions within the membrane. Finally, there are two conserved proline residues present in the transmembrane domain, suggesting that this region is arranged in a specific conformation. Together, these three lines of evidence indicate that the transmembrane domain of this class of receptor chaperons, e.g., UNC-74 or TMX3, is functionally significant for a function other than just membrane anchorage.

Many mammalian nAChR subunits contain a motif that is necessary and sufficient for ER retention (Wang et al., 2002; Wang et al., 1996). This motif PL(F/Y)(F/Y)XXN (a ER retention motif) is frequently present at the amino terminal end of the first transmembrane spanning region, however it is not clear whether this motif is within the lipid bi-layer of the membrane or located on the luminal side of the membrane (Unwin, 2005). While the precise location of the motif is not currently known, it is believed to play a role in retaining subunits in the ER until they are assembled into functional receptors, e.g., pentamer nAChRs. For example, once subunits of the nAChR are assembled into pentamers, the motif is believed to become buried and the mature pentamer is trafficked to the plasma membrane. This provides a means of regulation that ensures trafficking of only assembled subunits. The affects of mutation within this motif on the formation of functional receptors has not been fully determined, but alanine scanning mutagenesis indicated that perturbation of the motif results in the surface localization of individually expressed subunits that are normally retained in the ER. This motif is found in some, but not all nAChR subunits, e.g., levamisole sensitive nAChR subunits of C. elegans (Table 2). For example, UNC-39 contains this motif, but UNC-29, LEV-1, and UNC-63 have amino acid substitutions that disrupt this motif. UNC-29, LEV-1 and UNC-63 all have a threonine residue in place of the proline at the first position of this motif. In addition, both UNC-29 and LEV-1 contain a hydrophobic residue in place of the terminal asparagine. Similar amino acid changes are also found in mammalian α5 and β3 subunits. The absence of this motif in some, but not all, subunits of nAChRs suggest that some subunits are kept in the ER prior to assembly via their own ER retention motif, while the remaining subunits may require other factors to keep them in the ER prior to assembly. In the absence of such a factor, subunit that require such a factor, are trafficked to the cell surface prior to proper assembly into the receptor. Thereby, reducing or eliminating the function of the receptor.

TABLE 2
Sequence alignment of nAChR subunit ER retention motif.

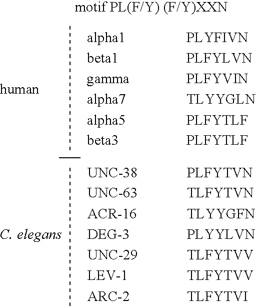

Sequence of ER retention motif from selected human and C. elegans nAChR subunits. Residues that deviate from the consensus motif are in red.

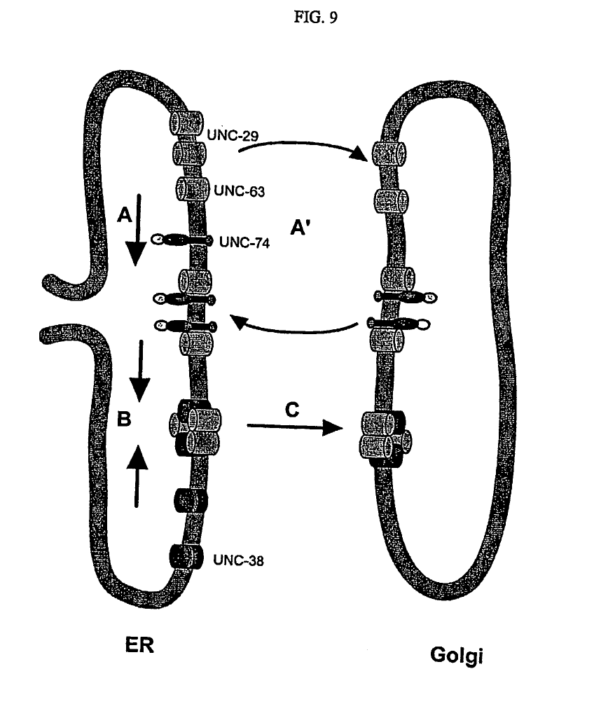

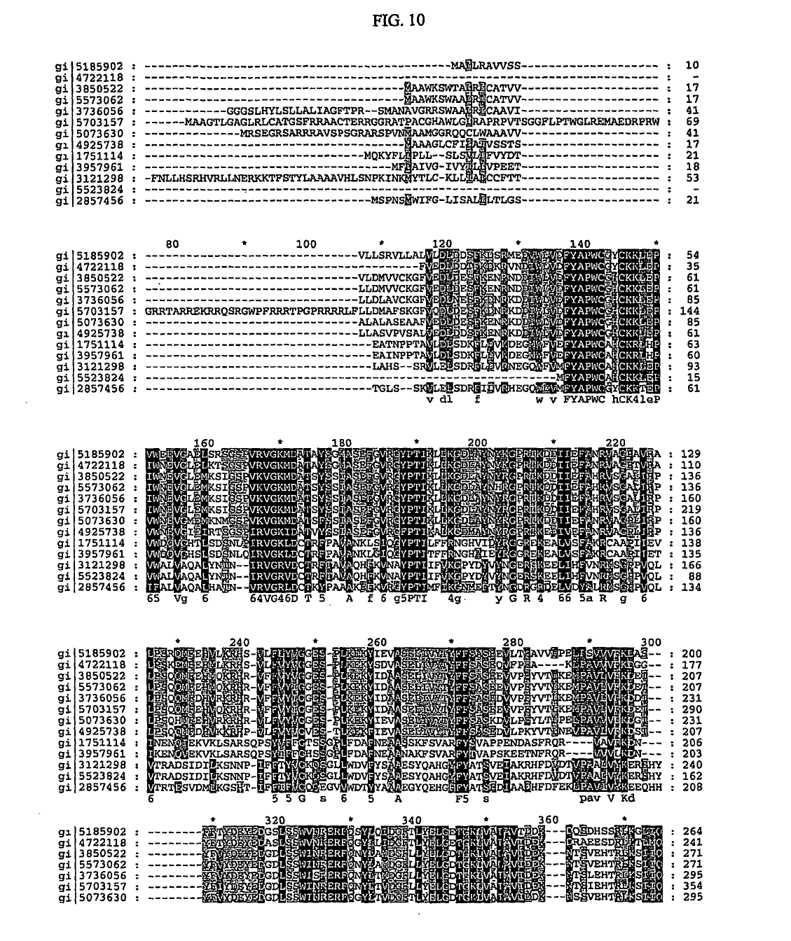

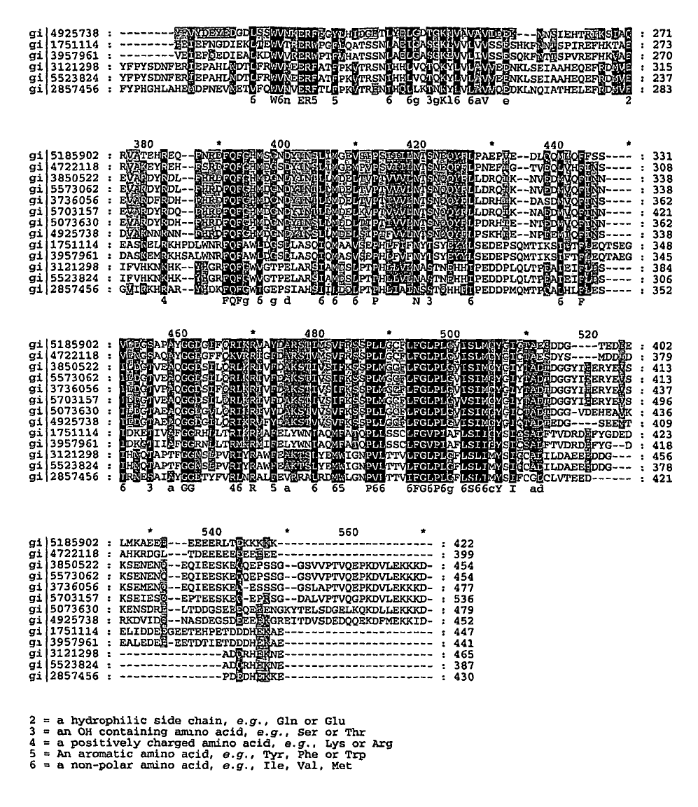

The present observations suggest that receptor chaperons, e.g., UNC-74 or TMX3, function as an exogenous ER retention factor that retains specific nAChR subunits in the ER. Thereby keeping subunits lacking the ER retention motif in the ER, and allowing assembly with other subunits kept in the ER via endogenous the ER retention motifs (FIG. 9). Therefore, UNC-74 is believed to function by keeping UNC-29, LEV-1 and UNC-63 in the ER, allowing them to form functional nAChR pentamers with UNC-38.

Without wishing to be bound by theory, UNC-74 is believed to keep these subunits in the ER through direct interaction between the UNC-74 transmembrane helix and transmembrane spanning regions of nAChR subunits. This may be accomplished two different ways. First, UNC-74 may remain in the ER interacting with unassembled nAChR subunits, thus keeping them in the ER until assembly. Alternatively, UNC-74 may shuttle between the ER and cis-Golgi, retrieving subunits that have escaped the ER. This model makes three readily testable predictions, one or more of which are tested. First, a functional UNC-74 transmembrane domain and an intact ER retention motif should be required for UNC-74 function. Second, subunits that lack the ER retention motif should not form, or have a reduced ability to form, dimers or higher order oligomers with other subunits in the absence of UNC-74. Finally, nAChR subunits that do not contain the ER retention motif should not be retained in the ER of unc-74 mutants and/or addition of this ER retention motif to subunits that do not have it should bypass the requirement for UNC-74 function. These models can be tested using the guidance of the present invention, in combination with established genetic and biochemical techniques.

Previously published reports on TMX3, the human homologues of UNC-74, were limited to characterization of predicted motifs in the protein and cellular localization, but did not mention a function for TMX3. Northern blot data indicated that TMX3 transcripts were very broadly, if not ubiquitously, expressed, but enriched in skeletal cardiac muscle. The enrichment in skeletal muscle is now consistent with the present invention, where TMX3 is believed to play a role in the formation of muscle type nAChR. Heterologous expression of mammalian muscle type nAChRs in Xenopus oocytes was used to test whether mammalian UNC-74 can increase the formation of Xenopus muscle type nAChR.

While no increase in native Xenopus nAChR expression, measured by two-electrode voltage clamp, was observed by the addition of the mouse homologue of UNC-74, the ubiquitous expression of TMX3 is consistent with Xenopus homologues of unc-74 being expressed in Xenopus oocytes, allowing the formation of vertebrate nAChRs. Therefore, the fact that expression of a mammalian UNC-74 homologue does not appear to increase production of native nAChRs in Xenopus oocytes is consistent with the proposed mechanism of action. However, expression of heterologous nAChR subunits (homomultimers and heteromultimeric forms) has been shown to be limited and expression of a complementary receptor chaperon, for example, a receptor chaperon derived from the same species as the nAChR subunit, may overcome the limited expression.

The model presented herein suggests that unc-74 homologues function in the promotion of nAChR subunits that do not contain the ER retention motif, including α7, α5 and β3 subunits. α7 subunits are notoriously difficult to express in certain cell lines. The low level of α7 homopentamer expression in these cell lines may be due to low-level expression or the complete lack of a complimentary UNC-74 homologue. Therefore, expression of a mammalian homologue in these cells may increase production of homopentamers. In addition, α5 subunits require the presence of another α subunit to be incorporated into pentamers. Interestingly, both UNC-63 and UNC-38 encode α-like subunits and are likely to be incorporated into the same channel. Therefore, the present data is believed to define a class of unc-74 homologues (e.g., orthologous) that have a similar function in other systems.

A model presented herein predicts that the UNC-74 transmembrane spanning region and ER retention motifs are necessary, and possibly sufficient, for unc-74 function. This prediction could be tested by generating a set of deletions and point mutations that disrupt the unc-74 transmembrane spanning region and ER retention motif. It is expected that expression of mutant constructs will fail to rescue the unc-74 mutant phenotype. Alternatively, unc-74 mutants could be rescued with just the luminal portion of UNC-74. This might indicate that unc-74 has a processing or folding role by acting on the extracellular region of nAChR subunits. If the carboxy terminal end of UNC-74 is found to be dispensable for function, then a noncomplementation screen could be done to isolate other alleles of unc-74 that may identify functionally important regions of the protein. Because levamisole resistance is a robust phenotype it should be easy to obtain a large panel of alleles, some of which may be due to point mutations in essential regions of the protein.

The model presented herein also hypothesizes that pentamers are not formed in unc-74 mutants because subunits that make up the pentamer quickly leave the ER prior to assembly. Biochemical techniques could be used to see whether pentamers are formed. In addition, co-immunoprecipitation could be used to determine whether subunits that contain the motif are in a complex with other subunits. An alternative to the model is that UNC-74 has a role in trafficking of assembled pentamers out of the ER. Immunoprecipiation of pentamers in unc-74 mutants would provide support for this alternative model.

The localization of UNC-38:GFP in an unc-74 mutant background established that UNC-38 is retained in the ER. However, the localization of other levamisole sensitive nAChR subunits that do not contain the motif has not been determined. The model predicts that subunits that do not contain the ER retention motif will not be localized to the ER in unc-74 mutants. This prediction could be tested by examining the cellular localization of GFP fusion proteins. The model presented herein hypothesizes that the direct target of UNC-74 is subunits without the ER retention motif. If this is the case, then restoration of the motif to UNC-29, UNC-63 and LEV-1 should bypass the requirement for unc-74. This proposition can be tested by replacing the motif of UNC-29, UNC-63 and LEV-1 with the intact motif of UNC-38 and assaying whether expression of these subunits rescue unc-74 mutants. If the model is correct, restoration of the motif will keep these subunits in the ER, allowing them to assemble in the absence of unc-74. It is possible that ER retention motif has functional role and that perturbation of the ER retention motif disrupts function, but not formation of nAChRs. If this were the case, then the phenotype of ER retention motif mutants would be identical to unc-74 mutants making interpretation of a negative result impossible. To get around this issue, GFP tagged subunits could be used and examined for proper localization in the absence of unc-74.

One feature of UNC-74 is the presence of homologues in other systems. All metazoans in which it has been found contain nAChRs and those organisms in which it has been shown to be absent (i.e., fungi) do not have nAChRs. UNC-74 may have a similar role in the formation of mammalian nAChRs that it has in C. elegans. To date, there is no evidence other than protein conservation to indicate that homologues of unc-74 have a role in nAChR formation. Expression of Munc-74 (the mouse homologue) in body wall muscles of unc-74 mutants failed to rescue the unc-74 mutant phenotype (data not shown). This could be due to lack of interaction between Munc-74 and worm nAChRs subunits or other unidentified proteins that require unc-74 function, perhaps unc-50. Previously published reports on TMX3, the human homologues of UNC-74, were limited to characterization and cellular localization and did not mention a function for TMX3. Northern blot data indicated that TMX3 transcripts were very broadly, if not ubiquitously, expressed but enriched in skeletal cardiac muscle. The enrichment in skeletal muscle suggests that TMX3 may have a role in the formation of muscle type nAChR. Heterologous expression of mammalian muscle type nAChRs in Xenopus oocytes was used to test whether mammalian UNC-74 has a role in the formation of mammalian muscle type nAChR. No increase in nAChR expression, measured by two electrode voltage clamp, was observed by the addition of Munc-74. The ubiquitous nature of TMX3 expression suggests that homologues of unc-74 may be expressed in Xenopus oocytes, allowing the formation of vertebrate nAChRs.

Reduction of expression of a receptor chaperon may be used to treat diseases. For example, in humans, the epidermal growth factor receptor (Egfr) belongs to a family of ErbB receptors. Hyperactive receptor signaling of these receptors has been linked to human cancers, including, but not limited to, brain, breast, lung, colon, and epidermis. For example, ErbB1 is frequently amplified in about 85% of squamous cell carcinomas and ErbB2 is frequently amplified in breast, stomach, and ovarian cancer. Hence, reducing the number of ErbB receptors, hyperactive receptors, may provide a method of treating such diseases. For example, RNAi may be used to reduce the expression of a receptor chaperon, or compounds that inhibit a receptor chaperon, may be used to reduce the number of receptors that are trafficked to the cell surface, thereby treating the disease.

As used herein, “receptor chaperon” means a protein or polypeptide that functions in the formation of a multimeric receptor on the surface of a cell, the protein or polypeptide may function during subunit processing and folding, subunit assembly, or trafficking of mature receptors. The receptors are preferably pentamers. As will be recognized by a person of ordinary skill in the art, the current use of the phrase “receptor chaperon” is not be limited to the activities of known chaperone proteins, however, the reader will also recognize common attributes between the present class of receptor chaperons and more “classical” chaperones. Thus, the term is not intended to limit the invention, but rather to provide a convenient descriptive term.

Any of the methods for studying ligand-gated ion channels known in the art, for example, see U.S. Patent Publication 20040132187 to Groppi et al. (Jul. 8, 2004), may be used in the invention. For the sake of brevity, descriptions of these methodologies, which include such things as RNAi, mutagenesis, cell culture, preparation an effective amount of a pharmaceutical, and the like, are omitted and the reader being referred to alternative descriptions, including, but not limited to, U.S. Patent Publications 20040224910, 20030153519, 20030175772, 20040203132, 20030138911, 20040185468, 20040248189, 20050033522, 20040132187, Ausubel, F. M. et al. (eds) (1997) CURRENT PROTOCOLS IN MOLECULAR BIOLOGY, John Wiley & Sons., Shimomurand et al. (2002) Effects of Mutations of a Glutamine Residue in Loop D of the α7 Nicotinic Acetylcholine Receptor on Agonist Profiles for Neonicotinoid Insecticides and Related Ligands, Br. J. Pharmacol. 137:162-169, REMINGTON′S PHARMACEUTICAL SCIENCES, 18th Ed. (1990, Mack Publishing Co., Easton, Pa.).

As used herein, “substantially pure” means a preparation which is at least 60% by weight (dry weight) of the compound of interest. Preferably, the preparation is at least 75%, more preferably at least 90%, and most preferably at least 99% by weight of the compound of interest. Purity can be measured by any appropriate method, for example, column chromatography, polyacrylamide gel electrophoresis, or HPLC analysis.

As used herein, an “isolated nucleic acid” means a nucleic acid that is not immediately contiguous with both of the coding sequences with which it is immediately contiguous (one on the 5′ end and one on the 3′ end) in the naturally occurring genome of the organism from which it is derived. The term therefore includes, for example, a recombinant nucleic acid which is incorporated into a vector; into an autonomously replicating plasmid or virus; or into the genomic DNA of a prokaryote or eukaryote, or which exists as a separate molecule (for example, a cDNA or a genomic DNA fragment produced by PCR or restriction endonuclease treatment) independent of other sequences. It also includes a recombinant nucleic acid which is part of a hybrid gene encoding additional polypeptide sequences.

As used herein, a “substantially identical” polypeptide sequence means an amino acid sequence which differs from a reference sequence only by conservative amino acid substitutions, for example, substitution of one amino acid for another of the same class (for example, valine for glycine, arginine for lysine, etc.) or by one or more nonconservative substitutions, deletions, or insertions located at positions of the amino acid sequence which do not destroy the function of the polypeptide (assayed, for example, as described herein). Preferably, such a sequence is at least 73%, more preferably at least 85%, and most preferably at least 95% substantially identical at the amino acid level to the sequence used for comparison. The invention encompasses polypeptide sequences being 73-99% substantially identical to the amino acid sequences set forth in any one of SEQ ID NO:9 through SEQ ID NO:27. Sequence identity is typically measured using sequence analysis software (for example, Sequence Analysis Software Package of the Genetics Computer Group, University of Wisconsin Biotechnology Center, 1710 University Avenue, Madison, Wis., 53705, or BLAST software available from the National Library of Medicine). Examples of useful software include the programs, PILE-UP™ and PRETTYBOX™. Such software matches similar sequences by assigning degrees of homology to various substitutions, deletions, substitutions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

As used herein, “an expression vector” means that the nucleic acid molecule of interest is operably linked to a sequence which directs transcription and/or translation of the nucleic acid molecule.

As used herein, “peptide,” “polypeptide” and “protein” are used interchangeably, no distinction, based on length, is intended between a peptide, a polypeptide or a protein.

### EXAMPLES

### Behavioral and Pharmacological Assays

Swimming assay. Well bottoms of a 96-well microtiter plate were layered with 50 μl of 2% agarose in M9. Individual young adult worms were suspended in 50 μl M9 in single wells. After a >5 minute recovery period, the number of complete body bends were counted for a minimum of 60 seconds. Each worm was assayed a minimum of three times and body bend frequency per worm is the average of all assays. Data presented is the mean +/− SEM of n=number of worms.

Pharmacological resistance assays. Plates were equilibrated to room temperature, weighed, and equal volumes of different concentrations of levamisole or aldicarb were added to make plates containing specific concentrations of drug. Once plates dried, they were seeded with a drop of OP50 and incubated overnight at room temperature. A minimum of 15 young adult animals (levamisole) or L4 animals (aldicarb) were placed on drug plates and maintained for two (levamisole) or eight (aldicarb) hours. The percentage of animals on each plate that were paralyzed in response to mild stimulation was determined. Data presented represents the mean +/− SEM of n=number of plates.

Locomotion assay. Unseeded plates were stained with 0.1% Bromophenol blue and seeded with a thin lawn of HB101. Four young adult animals of the same genotype were placed on plates and allowed to recover for 10 minutes. Digital movies were obtained of the entire plate, and the velocity each animal moved per one second frame was determined using Image J Worm Tracker 6. Overall mean velocity was calculated for each animal by averaging over a minimum of 500 frames. Data presented represents the mean +/− SEM for n=number of animals.

### Molecular Biology

Sequencing unc-74 mutant alleles. The molecular lesion of unc-74 alleles was determined by genomic amplification of the unc-74 locus from different strains using oligonucleotides oDW05 (5′-CAGATCACATAATAAGCCCGGAACC-3′; SEQ ID NO:1, of the incorporated herein Sequence Listing) and oDW06 (5′ CCATTCCTTATCGACGAGCCTTTGG-3′; SEQ ID NO:2). For each allele sequenced, the entire 3491 bp amplicon was directly sequenced at the University of Utah Sequencing Facility.

### Constructs Used

pJL29[UNC-38::GFP] Generated by Jean-Louis Bessereau. Contains GFP in the intracellular loop between TM3 and TM4 of UNC-38 (Jean-Louis Bessereau, personal communication).

mCTRAM[TRAM::CFP] Gift from M. Rolls and T. Rappaport. (Rolls et al., 2002).

pDW14[Punc-74:GFP] A 924 bp BamHI to NsiI fragment of the unc-74 promoter region cloned into the BamHI—PstI sites of pPD95.69.

pDW61[unc-74 cDNA] A 1364 amplicion, generated with oDW73 (5′-TCACTCGAGCTGGGTCACTCAGCTTTTTCGT-3′; SEQ ID NO:3) and oDW86 (5′-CAGGCTATGCAAAAATATTTCTTATTACC-3′; SEQ ID NO:4) using a cDNA clone (Vidal ORFeome clone) as template, blunt cloned into pCR-Blunt.

pDW80[Pmyo-3:unc-74cDNA] A 1454 by fragment generated by digestion of pDW61[unc-74cDNA] with XbaI and Acc65I, inserted into the NheI—Acc65I sites of the myo-3 promoter vector pPD95.62, from Fire Vector Kit.

pDW84[Pmyo-3:unc-74cDNA::GFP] A 945 by fragment from XmaI digestion of pPD 102.33 (GFP exon protein fusion, Fire Vector Kit), inserted into the BspEI site of pDW80[Pmyo-3:unc-74cDNA::GFP].

pDW88[Prab-3:unc-74cDNA] A 1267 by fragment generated ZraI—NotI digest of Prab-3:pGEMT (M. Hammarlund and K. Schuske) subclone, inserted in the PmlI—NotI sites of pDW80[Pmyo-3:unc-74cDNA]. This replaced the myo-3 promoter with the rab-3 promoter to drive the expression of the unc-74 cDNA.

pUNC-49B:GFP[UNC-49:GFP] Generated by Bruce Bamber. Contains the coding region of GFP between TM3 and TM4 of UNC-49B (B. Bamber, personal communication).

UNC-74 Thioredoxin Active Site Mutations

pDW85[Pmyo-3:unc-74(SAHC)]

pDW86[Pmyo-3:unc-74(CAHS)]

pDW87[Pmyo-3:unc-74(SAHS)]

A mutated thioredoxin active site amplicon was generated by PCR amplification using oDW43[SAHC] (5′-TACGCTCCATGGAGTGCTCACTGCAAGCGC-3′; SEQ ID NO:5), oDW44[CAHS] (5′-TACGCTCCATGGTGTGCTCACAGCAAGCGC-3′; SEQ ID NO:6), or oDW45[SAHS] (5′-TACGCTCCATGGAGTGCTCACAGCAAGCGC-3′; SEQ ID NO:6) as the upstream primer and oDW46 (5′-TTCACCGTCATCACCGAAACGCGCGAGG-3′; SEQ ID NO:7) using pDW80[Pmyo-3:unc-74cDNA] as template. The amplicons were cut with NcoI and ClaI and inserted into the NcoI—ClaI sites of pDW80[Pmyo-3:unc-74cDNA]. Verification of active site mutations was determined by sequencing

Immunocytochemistry and Confocal Microscopy.

Antibodies against SNB-1 were a gift from M. Nonet (Nonet et al., 1998). For immunocytochemistry worms were fixed in cold 2% paraformaldehyde and then cut into ˜0.1 mm transverse sections. Sections were washed in PBS, then decorated with primary anti-SNB-1 as previously described (Nonet et al., 1997). After washing, worm sections were treated with secondary goat anti-rabbit conjugated Alexa 568 antibodies (Molecular Probes). Worm sections were mounted on agarose pads and examined using a Bio-Rad Radiance Laser 2000 laser-scanning confocal microscope.

Intact double transgenic animals expressing UNC-74:GFP and TRAM:CFP were mounted on agarose pads and immobilized in 2% phenoxypropanol. Images were collected on a Zeiss Laser Scanning Microscope 5 PASCAL; GFP was excited using a 488 nm laser and emissions collected from 505-600 nm and CFP was excited at 405 nm and emissions collected from 420-480 nm. Transgenic animals with only one transgene were examined to ensure no bleed through of one fluorescent protein in the other channel.

Electrophysiology

Electrophysiology was performed as previously described (Richmond and Jorgensen, 1999a).

unc-74 Mutants Lack Functional Levamisole Sensitive nAChRs

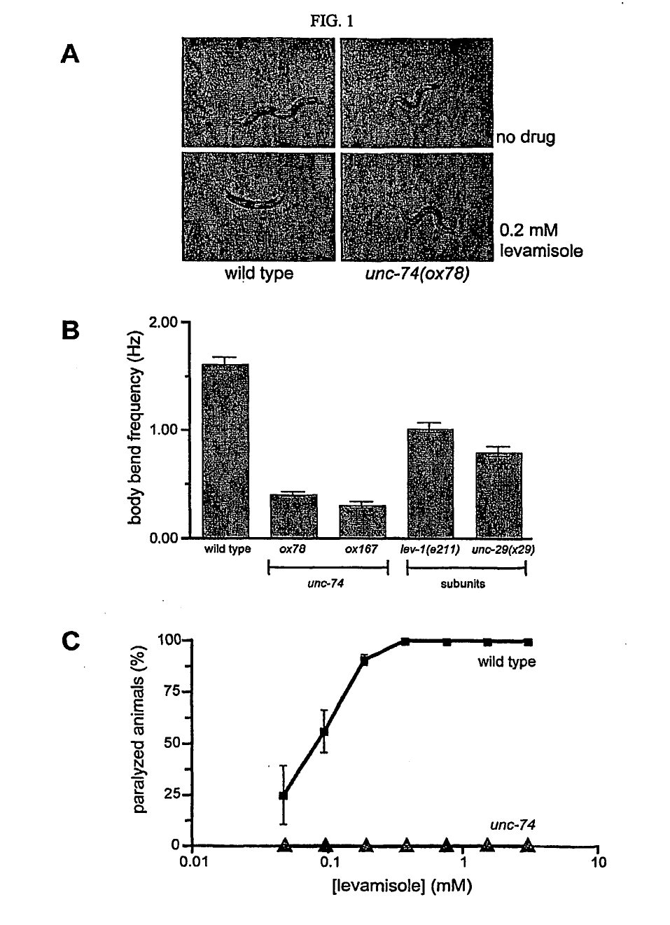

Mutations in unc-74 cause behavioral and pharmacological phenotypes that are characteristic of defects in cholinergic neurotransmission mediated by the levamisole sensitive nAChR (FIG. 1). Mutations in the levamisole sensitive nAChR subunit genes cause a characteristic body posture and sluggish uncoordinated locomotion phenotype as well as complete resistance to levamisole (Culetto et al., 2004; Fleming et al., 1997). The body posture and locomotion of unc-74 mutant animals is indistinguishable from mutations in levamisole sensitive nAChR subunit genes. In addition, unc-74 animals show no response to concentrations of levamisole that cause complete paralysis of wild type animals. The locomotion defect was quantified by determining the frequency of body bends made by individual animals suspended in liquid media. Both unc-74 and levamisole sensitive nAChR subunit mutants exhibit a reduced body bend frequency when compared to wild type animals. Interestingly, the body bend frequency of unc-74 animals was less than both lev-1 and unc-29 mutants (subunits of the levamisole sensitive nAChR).

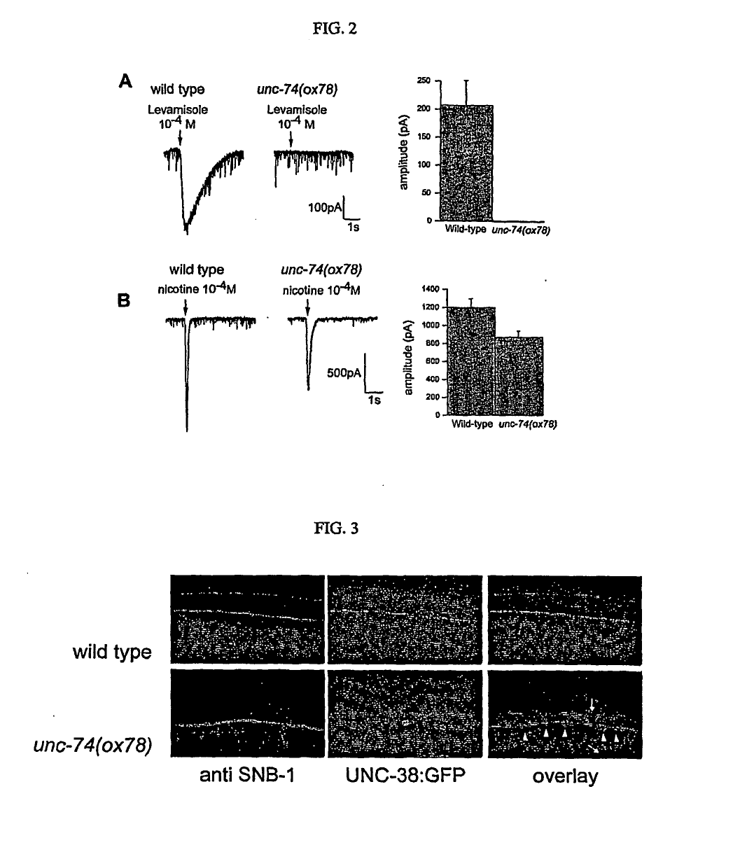

To directly examine levamisole sensitive nAChR function, electrophysiological analysis was performed on wild type and unc-74 mutant animals (FIG. 2). Intact body wall muscles of dissected animals were voltage clamped and the amount of current elicited in response to levamisole was measured. Focal pulses of levamisole to wild type muscle cells resulted in a robust inward current of around 200 pA. In contrast, levamisole induced current is completely abolished in unc-74 mutants. Importantly, miniature postsynaptic potentials were recorded from unc-74 muscles, indicating that lack of levamisole induced currents is not due to gross perturbation of synaptic function. In addition, a nicotine response is present in unc-74 animals, demonstrating that there are functional nAChRs present on the muscle cell surface of these animals. These results show that muscle cells of unc-74 animals do not have functional levamisole nAChRs on the plasma membrane.

nAChR subunits are retained in the ER.

The lack of functional levamisole sensitive nAChRs on the muscle cell surface could be due to a block in nAChR formation in unc-74 mutant animals. To examine this possibility further, the cellular localization of UNC-38, an α subunit of the levamisole sensitive nAChR, was determined (FIG. 3). The coding region of GFP was inserted into the intracellular loop between M3 and M4 of unc-38 and this fusion protein was expressed under its own promoter. Transgenic expression of UNC-38:GFP rescues the uncoordinated, levamisole resistance and electrophysiological defects of unc-38 mutations. In these animals, GFP signal is localized to discrete punctae along the dorsal and ventral nerve cords. These punctae are synaptic, since UNC-38:GFP localization is juxtaposed to the signal from antibody staining against the pre-synaptic marker SNB-1 (Nonet et al., 1998). In contrast, the rescuing UNC-38:GFP fusion protein was not synaptic in an unc-74 mutant background. Instead, fluorescence was present in neuronal cell bodies and concentrated around the nucleus of muscle cells in a diffuse pattern throughout the cell. Based on the localization of GFP signal in neuronal cell bodies and distribution of GFP throughout muscle cells, it appears that UNC-38 is retained in the ER of unc-74 mutant animals. This result is consistent with unc-74 functioning to promote nAChR formation.

The unc-74 locus encodes a product homologous to TMX3

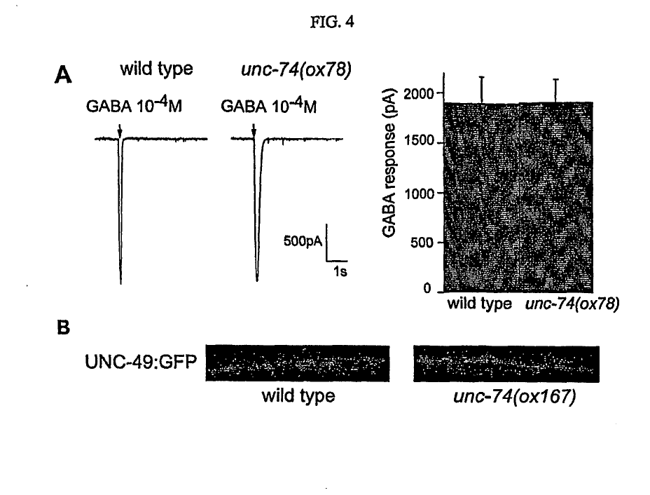

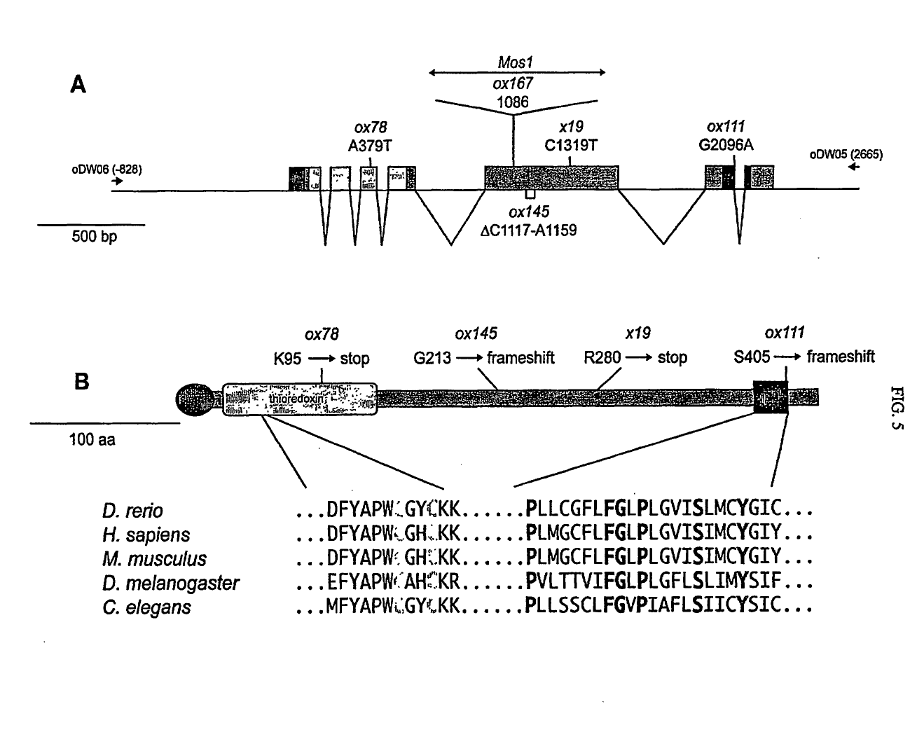

To address the question of how unc-74 is promoting formation of nAChRs, the unc-74 locus was cloned using Mos1 mediated mutagenesis (WO 00/73510; Bessereau et al., 2001; Williams et al., 2005). The F2 progeny of mutagenized animals were screened for animals with cholinergic neurotransmission defects using the acetylcholinesterase inhibitor aldicarb (Miller et al., 1996; Nguyen et al., 1995). From this screen one aldicarb resistant mutant, ox167, was isolated that was also uncoordinated and resistant to levamisole. Complementation testing demonstrated that ox167 failed to complement other alleles of unc-74, suggesting that ox167 is an allele of unc-74. A single Mos1 insertion in the fifth exon of the predicted gene ZK973.11 was tightly linked to the uncoordinated and drug resistance of unc-74(ox167). Germ line transformation rescue with a polymerase chain reaction (PCR) derived genomic fragment containing only the ZK973.11 open reading frame rescued the uncoordinated and drug resistance of ox167, as well as other alleles of unc-74 (data not shown). Finally the molecular lesions of many unc-74 alleles were determined by sequencing ZK973.11 obtained by PCR amplification using mutant genomic DNA as a template. Each mutant allele was due to a sequence change that is expected to disrupt UNC-74 protein function (FIG. 4).

Reverse-transcriptase PCR analysis and the sequence of expressed sequence tags were used to validate the predicted unc-74 gene structure (Reboul et al., 2001). The unc-74 coding region is 1344 by long and produces a mature protein product of 423 amino acids after cleavage of a 24 residue signal peptide. The amino terminal fourth of the protein is composed of a single thioredoxin domain. Thioredoxins and thioredoxin domains are prevalent in both prokaryotes and eukaryotes, where the primary biochemical activity is the formation and cleavage of disulfide bonds. This activity is dependent on a conserved C-X-Y-C active site that can be reversibly reduced or oxidized. The central UNC-74 region, comprising the majority of the protein, does not contain any identified functional domains, although there are two putative glycosylation sites. The carboxy terminal end contains a hydrophobic stretch that is predicted to be a transmembrane spanning region and a di-lysine like ER retention signal that is a hallmark of ER localized transmembrane protein. (Hardt and Bause, 2002). Based on these structural features, UNC-74 is believed to be a type I transmembrane protein with a thioredoxin domain, central domain, and to be retained in the ER lumen.

Thioredoxin domains are found in prokaryotes and eukaryotes, however, UNC-74 homologues make up a unique class of thioredoxin domain containing proteins. The central domain shares significant homology only with other proteins in this class. The central domain is not found in other thioredoxin domain containing proteins, for example, protein disulfide isomerases. UNC-74 homologues are predicted to have a signal peptide and transmembrane spanning region, as well as di-lysine ER retention signal. Homologues of UNC-74 have been found in other metazoans, but are conspicuously absent in fungi, which do not have a nervous system (no nAChRs). In addition to general protein organization and structural feature conservation, members of this unique class contain regions of sequence conservation identity outside of the thioredoxin domain. Intriguingly, the predicted transmembrane region contains significant sequence homology with polar resides, a conserved G-X-X-X-G motif, and multiple proline residues (Gerber et al., 2004; Niimura et al., 2005; Senes et al., 2004). Thus, the transmembrane region of UNC-74 is likely to have a function beyond just crossing the membrane. Recently a human homologue of UNC-74, called TMX3, was characterized and shown to be glycosylated and localized to the ER (Haugstetter et al., 2005). However, the cellular function of TMX3 was not established.

UNC-74 Functions in the ER of Muscle Cells.

Genes encoding subunits of the levamisole sensitive acetylcholine gated ion channel are expressed in both neurons and muscle cells (Culetto et al., 2004). However, expression of UNC-29 in muscle cells, expressed under the control of the myo-3 promoter, was sufficient to rescue unc-29 locomotion and levamisole resistance phenotypes (Fleming et al., 1997). This indicates that the levamisole resistance and uncoordinated phenotype is due to the lack of levamisole sensitive nAChRs in muscle cells. As shown above, UNC-38:GFP is retained in the ER of both neurons and muscle. To determine whether UNC-74 acts cell autonomously to promote formation of levamisole sensitive acetylcholine gated ion channels, the expression pattern of GFP under control of the unc-74 promoter was determined. In transgenic animals expressing this fusion protein, GFP signal was seen in neurons as well as body wall and head muscle cells (data not shown). This indicates that unc-74 is expressed in the same tissue types in which levamisole sensitive nAChR subunits are expressed and function. In addition, transgenes that express unc-74 in specific tissues were tested for phenotypic rescue (FIG. 5). Constructs were generated in which the unc-74 cDNA was placed under the control of the body wall muscle specific promoter, myo-3, or the pan neuronal promoter rab-3. unc-74 transgenic animals expressing unc-74 under control of these promoters were assayed for the locomotion and levamisole resistance unc-74 phenotypes. Muscle expression of unc-74 was able to rescue the unc-74 phenotype. In contrast, there was no difference in body bend frequency or levamisole resistance between unc-74 mutant animals and unc-74 mutant animals expressing unc-74 cDNA in neurons. These results demonstrate that unc-74 functions cell autonomously in muscle cells to promote nAChR formation.

UNC-74 is localized to the ER

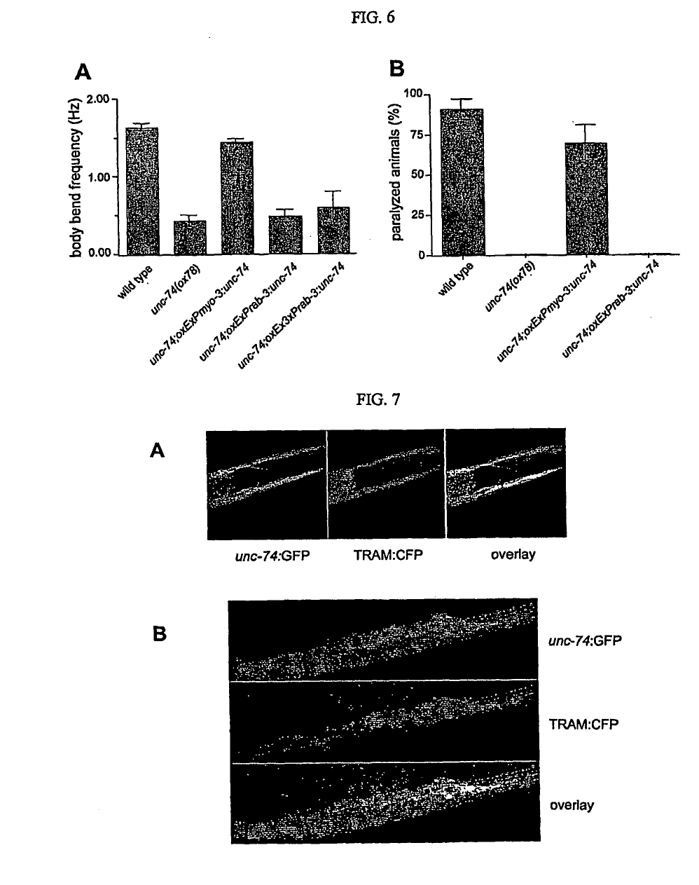

Analysis of UNC-74 protein sequence predicts that the mature protein will be localized to the ER. This prediction was tested by examination of the sub-cellular localization of a UNC-74:GFP fusion protein by confocal microscopy. The coding region of GFP was inserted in-frame into the carboxy terminal end of UNC-74, between the transmembrane spanning region and the putative ER localization signal. Expression of this transgene was able to rescue the unc-74 locomotion and levamisole resistance phenotypes (data not shown). In animals expressing UNC-74:GFP, signal was concentrated around the nucleus and diffuse throughout the muscle cell (FIG. 6). The UNC-74:GFP expression pattern is similar to UNC-38:GFP staining in unc-74 mutant animals. Verification that UNC-74:GFP is localized to the ER was demonstrated by comparing the localization of UNC-74:GFP with TRAM:CFP, an ER marker. Merged staked confocal images of animals expressing both UNC-74:GFP and TRAM:CFP show complete overlap of both fusion proteins. This result is consistent with the predicted ER localization of UNC-74 and further supports the ER localization of TMX3 in human cell lines.

UNC-74 Functions Independently of Redox Chemistry

A straightforward model for the mechanism by which UNC-74 promotes formation of nAChRs is that the thioredoxin domain of UNC-74 catalyzes formation of the Cys-loop on nAChRs subunits. Thus, the lack of unc-74 may be affecting nAChR formation by preventing formation of the disulfide bonds in a Cys-loop. This model would be consistent with the UNC-74 tissue and cellular localization data, which places the thioredoxin domain in the ER lumen, the site of Cys-loop formation. To test this model, unc-74 transgenes containing mutations in the thioredoxin active site were tested for in vivo function. Site-directed mutagenesis was used to mutate one or both of the unc-74 active site cysteine residues to serines. These constructs were introduced into unc-74 mutant animals and transgenic strains were assayed for rescue (FIG. 7). Unexpectedly, transgenic animals expressing active site mutations in which either or both cysteine residues had been changed were rescued for all unc-74 phenotypes assayed. There was no difference in the body bend frequency, velocity, or levamisole resistance between transgenic animals expressing wild type unc-74 and animals expressing active site mutant versions of unc-74. This result demonstrates that promotion of nAChR formation by UNC-74 does not require a functional thioredoxin domain. Therefore, UNC-74 is not believed to act catalytically in cystiene loop formation.

unc-74 is Specific for the Levamisole Sensitive nAChR

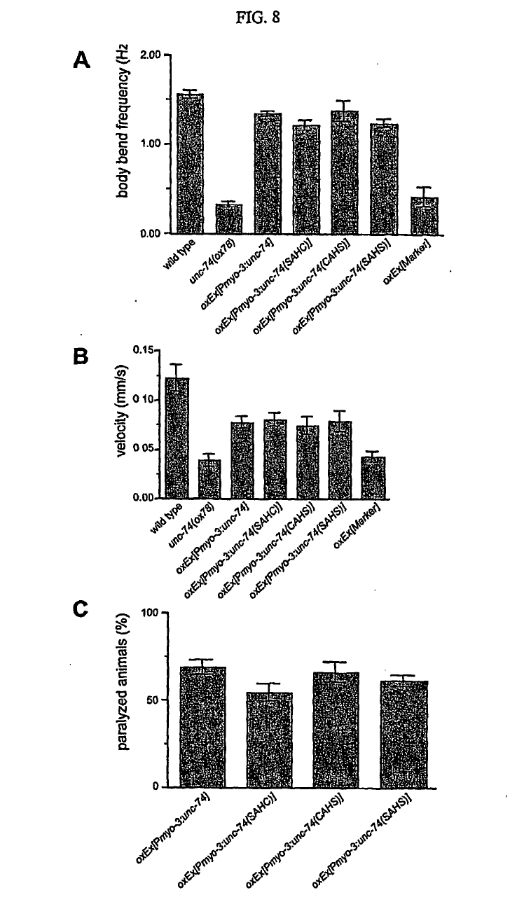

In addition to the levamisole sensitive nAChR, C. elegans expresses other Cys-loop channels. UNC-49 encodes a GABA_{A} receptor that is expressed in body wall muscles and EXP-1 is a cationic GABA gated channel expressed in enteric muscle and is required for the expulsion step of the defecation motor program (Bamber et al., 1999; Beg and Jorgensen, 2003; Richmond and Jorgensen, 1999a). In addition, the acr-16 locus encodes a Cys-loop, levamisole insensitive, nAChR (M. Francis et al., in press). Therefore, to determine if UNC-74 has a similar function in the formation of these related Cys-loop ligand gated ion channels, additional experiments were conducted. Five lines of evidence demonstrate that UNC-74 is specific for the levamisole sensitive nAChR (FIG. 8). First, electrophysiological analysis of unc-74 mutant animals resulted in GABA induced currents that were equivalent to GABA currents in wild type animals. Second, the localization of UNC-49/GABAa receptors were normal in unc-74 animals. Third, the frequencies of expulsions per defecation cycle were similar in unc-74 and wild type animals (data not shown). Fourth, unc-74 animals are only partially resistant to aldicarb and unc-74;unc-38 double mutants are no more resistant to aldicarb than either single mutant (data not shown). Finally, a nicotine-induced current is detected from voltage clamped unc-74 muscle cells. Although the amplitude of nicotine current is reduced in unc-74 animals relative to wild type, the reduction in current amplitude of unc-74 mutant animals is not greater than the reduction of nicotine-induced current amplitude seen in unc-38 mutants. This suggests that nicotine is also able to activate the levamisole sensitive nAChR. Together these results indicate that UNC-74 is a specific for the levamisole sensitive acetylcholine gated ion channel.

TMX3 Knockout in Human Cells

To examine the function of TMX3 on nAChR function in human cells, electrophysiological analysis is performed on differentiated PC12 cells transfected with an RNAi construct that inhibits expression of TMX3 (Meyer et al. (1998) Analysis of 3-(4-Hydroxy, 2-Methoxybenzylidene)Anabaseine Selectivity and Activity at Human and Rat Alpha-7 Nicotinic Receptors, J. Pharmacol. Exp. Ther. 287:918-925). After initial northern blot or western blot analysis to confirm the expression of TMX3, PC12 are exposed to either an RNAi construct specific to TMX3 or a negative control sequence. The cells are then voltage clamped and the amount of current elicited in response to various nAChR agonists and/or antagonists is measured. Pulses of a nAChR agonist or antagonist administered to the PC12 cells having the control RNA are found to produce an agonist dependent current or an antagonist dependent reduction in current. In contrast, the agonist induced current, or the antagonist dependent reduction, is decreased or abolished in PC12 cells where expression of TMX3 is reduced by the RNAi construct. These results will show that TMX3 functions in the production of functional nAChRs in PC12 cells.

Screening Compounds for Inhibition of a Receptor Chaperon

Differential expression of nAChR subunit genes from the AChR superfamily produces distinct receptor subtypes. Since each AChR subtype has a specific subunit composition, each subunit must contain some information leading to proper assembly. The neuronal AChR subunits α3 and α7 are presumably members of two different AChR subtypes. These subunits have different assembly behavior when expressed in heterologous expression systems: alpha 7 subunits are able to produce homomeric AChRs, whereas alpha 3 subunits require an additional factor(s) for functional expression of AChRs (Garcia-Guzman et al. (1994) Role of two acetylcholine receptor subunit domains in homomer formation and intersubunit recognition, as revealed by alpha 3 and alpha 7 subunit chimeras, Biochemistry 33(50):15198-203). This provides the ability to dissect the requirement for subunit interactions during AChR formation. Analysis of α7/α/3 chimeric constructs identified two regions essential to assembly and intersubunit recognition: an N-terminal extracellular region, a second domain within a region comprising the first putative transmembrane segment, M1, and the cytoplasmic loop coupling it to the pore-forming segment, M2, involved in the subsequent interaction and stabilization of the oligomeric complex (Id.).

Xenopus laevis oocytes are extracted from anesthetized females and placed in ND-96 medium (mM: NaCl 96, KCl 2, MgCl2 1, CaCl2·H2O 1.8, HEPES 5, Na-pyruvate 2.5, theophylline 0.5, and gentamicin, adjusted to pH 7.5). The oocyte clusters are incubated in 0.2% collagenase (type IA, Sigma-Aldrich) in ND-96 medium for defolliculation. Oocytes are agitated at 18.5° C. for 4 hours and then rinsed with Barth's medium (mM: NaCl88, KCl 1, NaHCO3 2.4, HEPES 15, pH 7.6). The oocytes are then left to recover for 24 h in oocyte medium, before injection of cDNA or RNA encoding TMX3. Appropriate amounts and ratios of cDNA or RNA are then injected into individual oocytes. The oocytes are then incubated at about 17° C. for about 1-2 days in ND-96 medium prior to injection of cDNA or RNA encoding nAChR subunits that are identified as requiring TMX3 for proper assembly. The oocytes may be precultured in the presence of potential inhibitors or the potential inhibitors may be added to the media after injection of the nucleic acid sequences encoding the nAChR subunits. After 1 to 2 days of incubation with the potential inhibitors, electrophysiology current recordings are made using whole oocytes. Recording electrodes preferably contain atropine to prevent muscarinic receptor stimulation and barium in place of calcium to avoid current amplification by calcium activated chloride currents (Coates, K. M. and Flood, P. (2001) Ketamine and its Preservative, Benzethonium Chloride, both Inhibit Human Recombinant 60 7 and α4β2 Neuronal Nicotinic Acetylcholine Receptors in Xenopus oocytes, Br. J. Pharmacol. 137:871-879). ACh is applied at a flow rate of approximately 4 ml min⁻¹ in about two second bursts. Oocytes, and the respective inhibitor, showing significant reductions in ACh triggered currents are identified.

### REFERENCES

Akabas, M. H., Kaufmann, C., Archdeacon, P., and Karlin, A. (1994). Identification of acetylcholine receptor channel-lining residues in the entire M2 segment of the alpha subunit. Neuron 13, 919-927.

Baker, R. E., Zwart, R., Sher, E., Millar, N. S. (2004). Pharmacological Properties of α9α10 Nicotinic Acetylcholine Receptors Revealed by Heterologous Expression of Subunit Chimeras. Mol. Pharmacol. 65, 453-460.

Bamber, B. A., Beg, A. A., Twyman, R. E., and Jorgensen, E. M. (1999). The Caenorhabditis elegans unc-49 locus encodes multiple subunits of a heteromultimeric GABA receptor. J Neurosci 19, 5348-5359.

Beg, A. A., and Jorgensen, E. M. (2003). EXP-1 is an excitatory GABA-gated cation channel. Nat Neurosci 6, 1145-1152.

Bessereau, J. L., Wright, A., Williams, D. C., Schuske, K., Davis, M. W., and Jorgensen, E. M. (2001). Mobilization of a Drosophila transposon in the Caenorhabditis elegans germ line. Nature 413, 70-74.

Brejc, K., van Dijk, W. J., Klaassen, R. V., Schuurmans, M., van Der Oost, J., Smit, A. B., and Sixma, T. K. (2001). Crystal structure of an ACh-binding protein reveals the ligand-binding domain of nicotinic receptors. Nature 411, 269-276.

Changeux, J. P., Devillers-Thiery, A., and Chemouilli, P. (1984). Acetylcholine receptor: an allosteric protein. Science 225, 1335-1345.

Connolly, C. N., and Wafford, K. A. (2004). The Cys-loop superfamily of ligand-gated ion channels: the impact of receptor structure on function. Biochem Soc Trans 32, 529-534.

Corringer, P. J., Le Novere, N., and Changeux, J. P. (2000). Nicotinic receptors at the amino acid level. Annu Rev Pharmacol Toxicol 40, 431-458.

Culetto, E., Baylis, H. A., Richmond, J. E., Jones, A. K., Fleming, J. T., Squire, M. D., Lewis, J. A., and Sattelle, D. B. (2004). The caenorhabditis elegans unc-63 gene encodes a levamisole-sensitive nicotinic acetylcholine receptor alpha subunit. J Biol Chem.

Davies, P. A., Wang, W., Hales, T. G., and Kirkness, E. F. (2003). A novel class of ligand-gated ion channel is activated by Zn2+. J Biol Chem 278, 712-717.

Dooley, C. T., Dore, T. M., Hanson, G. T., Jackson, W. C., Remington, S. J., and Tsien, R. Y. (2004). Imaging dynamic redox changes in mammalian cells with green fluorescent protein indicators. J Biol Chem 279, 22284-22293.

Fleming, J. T., Squire, M. D., Barnes, T. M., Tomoe, C., Matsuda, K., Ahnn, J., Fire, A., Sulston, J. E., Barnard, E. A., Sattelle, D. B., and Lewis, J. A. (1997). Caenorhabditis elegans levamisole resistance genes lev-1, unc-29, and unc-38 encode functional nicotinic acetylcholine receptor subunits. J Neurosci 17, 5843-5857.

Gerber, D., Sal-Man, N., and Shai, Y. (2004). Two motifs within a transmembrane domain, one for homodimerization and the other for heterodimerization. J Biol Chem 279, 21177-21182.

Green, W. N., and Wanamaker, C. P. (1997). The role of the cystine loop in acetylcholine receptor assembly. J Biol Chem 272, 20945-20953.

Green, W. N., and Wanamaker, C. P. (1998). Formation of the nicotinic acetylcholine receptor binding sites. J Neurosci 18, 5555-5564.

Hardt, B., and Bause, E. (2002). Lysine can be replaced by histidine but not by arginine as the ER retrieval motif for type I membrane proteins. Biochem Biophys Res Commun 291, 751-757.

Haugstetter, J., Blicher, T., and Ellgaard, L. (2005). Identification and characterization of a novel thioredoxin-related transmembrane protein of the endoplasmic reticulum. J Biol Chem 280, 8371-8380.

Imoto, K., Busch, C., Sakmann, B., Mishina, M., Konno, T., Nakai, J., Bujo, H., Mori, Y., Fukuda, K., and Numa, S. (1988). Rings of negatively charged amino acids determine the acetylcholine receptor channel conductance. Nature 335, 645-648.

Karlin, A., and Akabas, M. H. (1995). Toward a structural basis for the function of nicotinic acetylcholine receptors and their cousins. Neuron 15, 1231-1244.

Le Novere, N., and Changeux, J. P. (2001). LGICdb: the ligand-gated ion channel database. Nucleic Acids Res 29, 294-295.

Lewis, J. A., Wu, C. H., Berg, H., and Levine, J. H. (1980a). The genetics of levamisole resistance in the nematode Caenorhabditis elegans. Genetics 95, 905-928.

Lewis, J. A., Wu, C. H., Levine, J. H., and Berg, H. (1980b). Levamisole-resistant mutants of the nematode Caenorhabditis elegans appear to lack pharmacological acetylcholine receptors. Neuroscience 5, 967-989.

Merlie, J. P., Sebbane, R., Gardner, S., Olson, E., and Lindstrom, J. (1983). The regulation of acetylcholine receptor expression in mammalian muscle. Cold Spring Harb Symp Quant Biol 48 Pt 1, 135-146.

Miller, K. G., Alfonso, A., Nguyen, M., Crowell, J. A., Johnson, C. D., and Rand, J. B. (1996). A genetic selection for Caenorhabditis elegans synaptic transmission mutants. Proc Natl Acad Sci USA 93, 12593-12598.

Nguyen, M., Alfonso, A., Johnson, C. D., and Rand, J. B. (1995). Caenorhabditis elegans mutants resistant to inhibitors of acetylcholinesterase. Genetics 140, 527-535.

Niimura, M., Isoo, N., Takasugi, N., Tsuruoka, M., Ui-Tei, K., Saigo, K., Morohashi, Y., Tomita, T., and Iwatsubo, T. (2005). Aph-1 contributes to the stabilization and trafficking of the gamma-secretase complex through mechanisms involving intermolecular and intramolecular interactions. J Biol Chem 280, 12967-12975.

Nonet, M. L., Saifee, O., Zhao, H., Rand, J. B., and Wei, L. (1998). Synaptic transmission deficits in Caenorhabditis elegans synaptobrevin mutants. J Neurosci 18, 70-80.

Nonet, M. L., Staunton, J. E., Kilgard, M. P., Fergestad, T., Hartwieg, E., Horvitz, H. R., Jorgensen, E. M., and Meyer, B. J. (1997). Caenorhabditis elegans rab-3 mutant synapses exhibit impaired function and are partially depleted of vesicles. J Neurosci 17, 8061-8073.

Reboul, J., Vaglio, P., Tzellas, N., Thierry-Mieg, N., Moore, T., Jackson, C., Shin-i, T., Kohara, Y., Thierry-Mieg, D., Thierry-Mieg, J., et al. (2001). Open-reading-frame sequence tags (OSTs) support the existence of at least 17,300 genes in C. elegans. Nat Genet 27, 332-336.

Richmond, J. E., and Jorgensen, E. M. (1999a). One GABA and two acetylcholine receptors function at the C. elegans neuromuscular junction. Nat Neurosci 2, 791-797.

Richmond, J. E., and Jorgensen, E. M. (1999b). One GABA and two acetylcholine receptors function at the C. elegans neuromuscular junction. Nat Neurosci 2, 791-797.

Rolls, M. M., Hall, D. H., Victor, M., Stelzer, E. H., and Rapoport, T. A. (2002). Targeting of rough endoplasmic reticulum membrane proteins and ribosomes in invertebrate neurons. Mol Biol Cell 13, 1778-1791.

Schofield, C. M., Jenkins, A., and Harrison, N. L. (2003). A highly conserved aspartic acid residue in the signature disulfide loop of the alpha 1 subunit is a determinant of gating in the glycine receptor. J Biol Chem 278, 34079-34083.

Senes, A., Engel, D. E., and DeGrado, W. F. (2004). Folding of helical membrane proteins: the role of polar, GxxxG-like and proline motifs. Curr Opin Struct Biol 14, 465-479.

Sumikawa, K., and Gehle, V. M. (1992). Assembly of mutant subunits of the nicotinic acetylcholine receptor lacking the conserved disulfide loop structure. J Biol Chem 267, 6286-6290.

Unwin, N. (2005). Refined structure of the nicotinic acetylcholine receptor at 4A resolution. J Mol Biol 346, 967-989.

Williams, D. C., Boulin, T., Ruaud, A. F., Jorgensen, E. M., and Bessereau, J. L. (2005). Characterization of Most-Mediated Mutagenesis in Caenorhabditis elegans: A Method for the Rapid Identification of Mutated Genes. Genetics 169, 1779-1785.

All references, including publications, sequence identifiers, patents, and patent applications, cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A receptor chaperon comprising the polypeptide of SEQ ID NO:9.

2. A receptor chaperon comprising the polypeptide encoded by a nucleic acid sequence comprising SEQ ID NO:10.

3. A receptor chaperon, comprising the polypeptide of SEQ ID NO:12, 11, 13, 14, 15, 16, 17, 18, 19, 20, 21, 22, 23, 24, or 25.

4. The receptor chaperon of claim 1, wherein the receptor chaperon comprises a mutation in a thioredoxin domain.

5. The receptor chaperon of claim 4, wherein the mutation comprises changing a cysteine residue to a serine residue.

6. A method of producing a heterologous receptor in a cell, the method comprising:
providing a host cell;
introducing a nucleic acid sequence encoding at least one subunit of a heterologous receptor that does not efficiently produce a functional receptor on the surface of the host cell;
producing the at least one subunit of a receptor in the host;
introducing a nucleic acid sequence encoding a receptor chaperon of claim 1 into the host cell;
producing the receptor chaperon in the host cell; and
increasing production of a functional receptor comprising the at least one subunit of a heterologous receptor on the surface of the host cell.

7. The method according to claim 6, wherein the receptor chaperon comprises the polypeptide encoded by a nucleic acid sequence comprising SEQ ID NO:10.

8. The method according to claim 6, wherein the receptor chaperon comprises UNC-74, SEQ ID NO:12.

9. The method according to claim 6, wherein the receptor chaperon comprises TMX3.

10. The method according to claim 6, wherein the receptor chaperon comprises a mutation in a thioredoxin domain.

11. The method according to claim 10, wherein the mutation comprises changing a cysteine residue to a serine residue.

12. The method according to claim 6, wherein the at least one subunit of a heterologous receptor comprises a nicotinic acetylcholine receptor subunit.

13. The method according to claim 12, comprising a mammalian nicotinic Acetylcholine receptor subunit.

14. The method according to claim 13, wherein the nicotinic acetylcholine receptor subunit and receptor chaperon are obtained from the same mammalian species.

15. The method according to claim 14, wherein the receptor chaperon comprises TMX3.

16. The method according to claim 12, comprising a C. elegans nicotinic Acetylcholine receptor subunit.

17. The method according to claim 12, wherein the receptor chaperon comprises SEQ ID NO:12.

18. A method of reducing or eliminating expression of a receptor on a cell surface, the method comprising:
inhibiting a function of a receptor chaperon of claim 1 in a cell.

19. The method according to claim 18, further comprising inhibiting the function of the receptor chaperon in a subject thought to suffer from a disease characterized in that receptor hyperactivity is believed to be a causative agent of the disease or to produce undesirable effects in the subject.

20. An isolated nucleic acid sequence encoding a polypeptide comprising the amino acid sequence of SEQ ID NO:12.

21. The isolated nucleic acid sequence of claim 20, wherein the nucleic acid sequence comprises a cDNA sequence.

22. A vector comprising an isolated nucleic acid sequence encoding the receptor chaperon of claim 1, claim 2 or claim 3.

23. An expression vector comprising an isolated nucleic acid sequence encoding the receptor chaperon of claim 1, claim 2 or claim 3.

24. A host cell transformed by the vector of claim 22 or claim 23.

25. The host cell of claim 24, wherein the host cell is an immortalized cell line.

26. A method of producing a recombinant nematode nicotinic acetylcholine receptor, comprising culturing the host cell of claim 24 under conditions that permit the expression of UNC-74.

27. The method according to claim 26, wherein the unc-74 gene is coexpressed with one or more nAChr subunits.

28. A method of screening for anthelmintic compounds, the method comprising:
introducing a receptor chaperon of claim 1, claim 2 or claim 3 into a host cell;
exposing the host cell to a compound to be screened for anthelmintic activity;
selecting a compound which interacts with said receptor chaperon; and
characterizing the selected compound as an anthelmintic compound.

29. A method of controlling parasitic nematode growth in a host, the method comprising:
administering an effective amount of the anthelmintic compound identified in claim 28 to the host.

30. A method of controlling parasitic nematode growth in soil or a crop, the method comprising:
administering an effective amount of the anthelmintic compound identified in claim 28 to the soil or crop.
